(19) United States
(12) Patent Application Publication
(10) Pub. No.: US 2019/0307799 A1
(43) Pub. Date: Oct. 10, 2019

(54) ENGINEERED LYMPHOCYTES

(71) Applicants: The Regents of the University of Michigan, Ann Arbor, MI (US); Baylor College of Medicine, Houston, TX (US)

(72) Inventors: Challice Bonifant, Ann Arbor, MI (US); Stephen Gottschalk, Waco, TX (US)

(21) Appl. No.: 16/335,637

(22) PCT Filed: Sep. 22, 2017
(86) PCT No.: PCT/US17/52989

§ 371 Date: Mar. 21, 2019

Related U.S. Application Data
(60) Provisional application No. 62/398,859, filed on Sep. 23, 2016.

(51) Int. Cl. A61K 35/17; A61P 35/02; C07K 16/46; C07K 16/28; C07K 14/725; C12N 5/0783; C07K 14/705

(52) U.S. Cl. CPC A61K 35/17; A61K 2039/505; C07K 16/468; C07K 16/2851; C07K 16/2809; C07K 14/7051; C12N 5/0636; C07K 16/2866; C07K 14/70521; C07K 2317/31; C07K 2317/622; C07K 2319/03; C07K 2319/02; C07K 2319/30; A61P 35/02

(57) ABSTRACT

Provided here are compositions comprising engineered lymphocytes that secrete bispecific engager molecules, thereby activating T cells in the local environment to kill target cells. In particular embodiments, engineered lymphocytes selectively target CLL-1 positive leukemic cells, both directly and through activation of a subject's own T cells, while sparing CLL-1 negative cells, such as myeloid progenitor cells. In particular embodiments, engineered lymphocytes selectively target CD123 and CLL-1 positive leukemic cells, both directly and through activation of native T cells.

### CROSS-REFERENCE TO RELATED APPLICATIONS

The present invention claims priority to U.S. Provisional Patent Application 62/398,859, filed Sep. 23, 2016, which is incorporated by reference in its entirety.

### FIELD

Provided here are compositions comprising engineered lymphocytes that secrete bispecific engager molecules, thereby activating T cells in the local environment to kill target cells. In particular embodiments, engineered lymphocytes selectively target CLL-1 positive leukemic cells, both directly and through activation of a subject's own T cells, while sparing CLL-1 negative cells, such as myeloid progenitor cells. In particular embodiments, engineered lymphocytes selectively target CD123 and CLL-1 positive leukemic cells, both directly and through activation of native T cells.

### BACKGROUND

AML, with a cure rate of less than 30% for high risk and relapsed disease, is a cancer in need of new therapeutic strategies to impact patient survival. Despite the dismal prognosis, the mortality rate due to acute myeloid leukemia (AML) has remained relatively unchanged for the last two decades. The standard chemotherapy regimen has not been altered in nearly 40 years, in part because of the difficulty in specifically targeting leukemic myeloblasts while sparing normal tissue.

### SUMMARY

Provided here are compositions comprising engineered lymphocytes that secrete bispecific engager molecules, thereby activating T cells in the local environment to kill target cells. In particular embodiments, engineered lymphocytes selectively target CLL-1 positive leukemic cells, both directly and through activation of a subject's own T cells, while sparing CLL-1 negative cells, such as myeloid progenitor cells.

In some embodiments, provided herein are methods of treating acute myeloid leukemia (AML) in a subject, the method comprising adoptively transferring engineered lymphocytes that express a polypeptide construct comprising an antigen-recognition domain and an activation domain, wherein the antigen-recognition domain binds C-type lectin-like molecule-1 (CLL-1) displayed on a malignant myeloblast cell, and wherein the activation domain triggers an immune response against the malignant myeloblast cell by the engineered lymphocytes and/or native T cells upon binding of the antigen-recognition domain to CLL-1. In some embodiments, the polypeptide construct is a bispecific engager molecule which is secreted from the engineered lymphocytes. In some embodiments, the antigen-recognition domain is an antibody fragment that binds CLL-1. In some embodiments, the antibody fragment that binds CLL-1 is a single chain variable fragment (scFv). In some embodiments, the activation domain is a molecular moiety that interacts with T cell receptor (TCR) and induces an immunomodulatory signal. In some embodiments, the activation domain is an antibody fragment that binds CD3. In some embodiments, the antibody fragment that binds CD3 is a single chain variable fragment (scFv). In some embodiments, the polypeptide construct is a single polypeptide bispecific engager molecule comprising an scFv activation domain that binds CD3 and an scFv antigen-recognition domain that binds CLL-1. In some embodiments, the polypeptide construct is a chimeric antigen receptor (CAR) which is displayed on the surface of the engineered lymphocytes, and wherein the antigen-recognition domain and the activation domain are linked by a transmembrane domain. In some embodiments, the antigen-recognition domain is an antibody fragment that binds CLL-1. In some embodiments, the antibody fragment that binds CLL-1 is a single chain variable fragment (scFv). In some embodiments, the activation domain is an intracellular signaling domain that initiates signal transduction to activate the engineered lymphocyte upon antigen binding by the antigen-recognition domain. In some embodiments, the activation domain comprises a cytoplasmic sequence of the T cell receptor (TCR) and or co-receptors. In some embodiments, the engineered lymphocytes are engineered T-cells, engineered NK cells, or engineered NKT cells. In some embodiments, methods further comprise obtaining lymphocytes from the subject, genetically engineering the lymphocytes to express the polypeptide construct, and culturing the resulting engineered lymphocytes.

In some embodiments, provided herein are bispecific engager molecules comprising: (a) an antigen-recognition domain that specifically binds to C-type lectin-like molecule-1 (CLL-1); and (b) an activation domain that interacts with a portion of T cell receptor (TCR) to induce an immunomodulatory signal. In some embodiments, the antigen-recognition domain is an antibody fragment. In some embodiments, the antigen-recognition domain is a single chain variable fragment (scFv). In some embodiments, the activation domain is an antibody fragment. In some embodiments, the activation domain is a single chain variable fragment (scFv). In some embodiments, the activation domain is an anti-CD3 antibody fragment. In some embodiments, the activation domain and antigen-recognition domain are single chain variable fragments tethered to each other by a linker domain.

In some embodiments, provided herein are polynucleotides encoding the bispecific engager molecules described herein.

In some embodiments, provided herein are engineered lymphocytes comprising a polynucleotide that encodes a bispecific engager molecule described herein. In some embodiments, the engineered lymphocyte expresses the bispecific engager molecule from the polynucleotide and the bispecific engager molecule is secreted from the engineered lymphocyte. In some embodiments, the engineered lymphocyte is a T cell. In some embodiments, the engineered lymphocyte displays a molecular moiety on its surface that is a component of T cell receptor (TCR) or interacts with TCR to induce an immunomodulatory signal, and wherein the activation domain of the bispecific engager molecule is capable of binding the molecular moiety. In some embodiments, the engineered lymphocyte is an NK cell. In some embodiments, the NK cell expresses a chimeric antigen receptor (CAR) that comprises an antigen-recognition domain that is capable of binding to a cancer cell antigen. In some embodiments, the antigen-recognition domain of the CAR is capable of binding to CD123. In some embodiments, the CAR comprises in intracellular signaling domain that activates an immunomodulatory signal upon binding of the antigen-recognition domain of the CAR is capable of binding to CD123.

In some embodiments, provided herein are adoptive transfer methods comprising administering an engineered lymphocyte described herein to a subject. In some embodiments, the subject suffers from cancer. In some embodiments, the subject suffers from leukemia. In some embodiments, the subject suffers from acute myeloid leukemia (AML). In some embodiments, the engineered lymphocyte expresses and secretes the bispecific engager molecule within the subject. In some embodiments, the bispecific engager molecule binds to malignant cells displaying CLL-1 and T cells, thereby activating the T cells to attack the malignant cells displaying CLL-1.

In some embodiments, provided herein are engineered lymphocytes comprising: (a) a first polynucleotide sequence encoding bispecific engager molecule that comprises an antigen-recognition domain capable of binding a first antigen and an activation domain capable of binding a molecule moiety displayed on T cells that activates an immunomodulatory signal upon binding; and (b) a second polynucleotide sequence encoding a chimeric antigen receptor (CAR) that comprises an antigen-recognition domain capable of binding a second antigen and an intracellular signaling domain that activates an immunomodulatory signal upon binding of the antigen-recognition domain to the second antigen, wherein the intracellular signaling domain and the antigen-recognition domain of the CAR are linked by a transmembrane domain. In some embodiments, the first polynucleotide sequence and the second polynucleotide sequence are portions of a single nucleic acid or vector. In some embodiments, the first polynucleotide sequence and the second polynucleotide sequence are portions of separate nucleic acids or vectors. In some embodiments, the engineered lymphocyte is a T cell. In some embodiments, the activation domain of the bispecific engager molecule is capable of initiating primary immunomodulatory activation through T cell receptor, and the intracellular signaling domain of the CAR initiates a co-stimulatory immunomodulatory signal. In some embodiments, engineered lymphocyte is an NK cell. In some embodiments, the activation domain of the bispecific engager molecule is capable of initiating primary immunomodulatory activation through T cell receptor, and the intracellular signaling domain of the CAR initiates primary immunomodulatory activation of the NK cell. In some embodiments, the first antigen-recognition domain is capable of binding CLL-1 and the second antigen-recognition domain is capable of binding CD123. In some embodiments, the first antigen-recognition domain is capable of binding CD123 and the second antigen-recognition domain is capable of binding CLL-1. In some embodiments, methods are provided comprising administering an engineered lymphocyte described herein to a subject. In some embodiments, the subject suffers from cancer. In some embodiments, the subject suffers from leukemia. In some embodiments, the subject suffers from acute myeloid leukemia (AML).

In some embodiments, provided herein is the use of an engineered lymphocyte described herein for the treatment of a disease or condition. In some embodiments, the disease or condition is cancer. In some embodiments, the disease or condition is leukemia. In some embodiments, the disease or condition is acute myeloid leukemia (AML).

### BRIEF DESCRIPTION OF THE DRAWINGS

FIG. 1. Demonstration that CLL-1 is expressed on a range of human myeloid leukemia cell lines and primary acute myeloid leukemia samples. (A) FACS analysis of established KG1a, MOLM-13, MV-4-11, and OCI-AML-3, human myeloid leukemia cell lines. Anti-human CLL-1-FITC (clone: 50C1, BD Pharmingen) and isotype (shaded, mouse IgG1k, eBioscience)). (B) Primary human acute myeloid leukemia samples #1-5 express CLL-1. Anti-human CLL-1-FITC, fold increase of MFI over isotype.

FIG. 2. Demonstration that human T cells can be transduced to express bispecific engager specific for CLL-1 and CD3c (CLL1-ENG). (A) Schematic of retroviral vector expressing CLL1-ENG. IRES: Internal ribosomal entry site. mO: mOrange fluorescent protein. (B) FACS analysis for mOrange expression following representative transduction. (C) FACS analysis for mOrange expression following CLL1-ENG transduction of T cells isolated from 3 healthy donors. Non-transduced T cells used as control. (mean transduction efficiency: 81%, range: 76-89%)

FIG. 3. Demonstration of CLL1-ENG T cell activation and functional activity versus CLL-1-positive target cells. (A) CLL1+AML cell lines (MOLM-13, MV-4-11, OCI-AML-3) and CLL-AML cell line (KG1a) were cultured with non-transduced or CLL1-ENG T cells at an effector to target ratio of 1:1. 24 hours after culture, supernatant was isolated and tested for the presence of Interferon gamma (IFNγ, n=3 T cell donors). AML cells without T cells and T cells without target cells were included as control populations. (B) Co-culture experiments conducted and supernatant tested for Interleukin-2 (IL-2, n=3 T cell donors). (C) AML cell lines modified to stably express GFP.ffLuc were cultured with non-transduced or CLL1-ENG T cells at an effector:target ratio of 1:10. 7 days following induction of co-culture, presence of live AML cells was measured with luciferase assay. (D) CLL-1+MOLM-13.ffLuc AML cell line plated with non-transduced or CLL1-ENG T cells at E:T ratio of 1:10. 24 hours and 3 days after plating, presence of live cells measured with luciferase assay. (E) FACS plots of MV-4-11 and T cell co-culture 8 days after plating.

FIG. 4. Demonstration of CLL1-ENG activation of autologous primary T cells versus acute myeloid leukemia. (A) Peripheral blood samples (including primary leukemia and CD3+ T cells) were plated in the bottom of a tissue culture plate. CLL1-ENG T cells were plated in a 0.4 uM pore transwell. Seven days after plating, blast percentage was measured with FACS analysis (CD45med, SShi, and CD117+). No T cells and non-transduced T cells were plated in transwell as a negative controls. Blast percentage normalized to condition with no T cells in transwell.

FIG. 5. Demonstration of CD123-CAR expression. FACS analysis performed following retroviral transduction of activated T-cells with retroviral construct containing CD123 scFv, CD28 co-stimulatory region, and CD3ζ intracellular signaling domain.

FIG. 6. Demonstration of CD123-CAR T cell activation and cytotoxicity versus CD123+ target cells. (A) CD123+AML cell lines (MOLM-13, primary AML) and CD123-AML cell line (K562) were cultured with non-transduced or CD123-CAR T cells at an effector to target ratio of 1:1. 24 hours after culture, supernatant was isolated and tested for the presence of Interferon gamma (IFNγ, n=1 T cell donor). T cells without target cells were included as control populations. (B) Co-culture experiments conducted and supernatant tested for Interleukin-2 (IL-2, n=1 T cell donors). (C) MOLM-13 CD123+CD33+ cell line cultured with non-transduced or CD123-CAR T cells at an effector:target ratio of 1:1. 24 hours following induction of co-culture, presence of live AML cells was measured with FACS analysis for cells expressing the myeloid antigen CD33.

FIG. 7. Utility of CD123 and CLL-1 targeting. A. Antigen expression on AML cell lines and primary AML samples. Fold increase as compared to isotype control. B. Representative FACS plot of primary sample AML#3. C. Gating strategy to identify primary blasts.

FIG. 8. A schematic depicting the immunomodulatory action of exemplary (A) CD123-CAR-T cells, (B) CD123-ENG T cells, and (C) combinatorial CD123-CAR/CLL1-ENG T cells.

FIG. 8. CLL-ENG activity in vivo. NOD.Cg-Prkdc^{scid}Il2rg^{tm1Wj1}/SzJ mice (NSG, The Jackson Laboratory, Sacramento, Calif.) were administered an i.v. tail vein injection of 1e6 MV-4-11.ffLuc leukemia cells on DO. On D7 they were given no treatment, 10e6 control ENG-T cells, or 10e6 CLL-ENG T cells i.v. via tail vein. Leukemia progression was monitored with detection of whole bioluminescence per mouse and analyzed with IVIS software (Perkin Elmer, Waltham, Mass.). Median survival of mice without treatment: 50 days (n=5), with control-ENG T cells: 63 days (n=5), CLL-ENG T cells: 80.5 days (n=10).

FIG. 9A-D. Co-expression of CLL-ENG and CD123-CAR in T cells. T cells were transduced with retroviral vectors containing either CLL-ENG paired with the flurophore mOrange, or a CD123 chimeric receptor paired with the fluorophore ZsGreen. Successful genetic modification was evaluated with FACS analysis and detection of mOrange and/or ZsGreen. A. Unmodified T cells, B. CLL-ENG T cells, C. CD123-CAR T cells, D. CLL-ENG.CD123-CAR T cells. Red circle represents dual modified cells

FIG. 10. Enhanced activation with dual T cell modification. Modified T cells and target (CD123-negative/CLL1-negative or CD123positive/CLL1-positive) were plated in co-culture with unmodified, control-ENG, CLL-ENG, CD123-CAR, or CLL-ENG/CD123-CAR T cells. After 24 hours of culture, supernatant was harvested and assessed for the presence of Interferon-gamma as a marker of T cell activation. Experiment performed in technical duplicate with two unique T cell donors.

FIG. 11. Lack of toxicity to stem and progenitor hematopoietic cells by CLL1-targeting. Bone marrow mononuclear cells (BMMC) were isolated from healthy donor bone marrow via a density gradient. BMMC were plated with T cells (unmodified (NT), CD19-ENG, CLL-ENG, and CD123-ENG) at a 5:1 T cell to BMMC ratio and incubated for six hours at 37 degrees Celcius. After incubation, cells were plated in semisolid media containing growth factors (MethoCult H4434, Stem Cell Technologies, Cambridge, Mass.) and incubated for 12 days. Colonies were counted manually, identified as CFU-E or CFU-GM and count was then normalized to that of identical conditions without T cells present. (n=2 unique bone marrow donors and n=2 T cell donors. Each bone marrow challenged with each T cell donor, ns-non-significant difference, ****p<0.0001, **p<0.01).

FIG. 12. Demonstration of CAR-NK modification. Healthy donor NK cells were selected from peripheral blood mononuclear cells and transduced with retroviral vectors encoding chimeric receptors. Receptors contain an ectodomain to bind CD123 and an endodomain consisting of a combination of: TCRζ, DAP10, DAP12, 41BB, and/or CD28. CAR expression measured on D5 and D18 post-transduction with FACS analysis. Mean % CAR+ cells D5, D18: 41BB.ζ−70.6, 81.3; CD28.ζ−71 (D5 only); DAP10.41BB−85, 73; DAP12.41BB−68.1, 73.2.

FIG. 13A-B. Demonstration of specific NK-cell activation. A. NK cells and target (Raji (CD123-negative, MV4; 11 and Molm-13 (CD123-positive)) were plated in co-culture with unmodified, 41BB.ζ, DAP10.41BB, or DAP12.41BB CAR-NK cells. After 24 hours of culture, supernatant was harvested and assessed for the presence of Interferon-gamma as a marker of NK cell activation. Experiment performed in technical duplicate with 1-3 unique NK cell donors. B. NK cells and target (stably expressing firefly luciferase) were plated at a 1:2 effector:target ratio and cultured for 18 hours. BLI was then measured as a correlate to living cells in culture. Cytoxicity was then calculated as a comparison between cells identically treated without NK cells present. No difference was seen in cytoxicity of unmodified NK or NK-CAR for Raji (CD123-negative). Significantly increased killing (p<0.0001) was evident when comparing unmodified to CAR-NK for Molm-13 (CD123-positive).

FIG. 14. Demonstation of in vivo activation of NK-CAR. NOD.Cg-Prkdc^{scid}Il2rg^{tm1Wj1}/SzJ mice (NSG, The Jackson Laboratory, Sacramento, Calif.) were administered an i.v. tail vein injection of 1e6 MV-4-11.ffLuc leukemia cells on DO. On D7 they were given no treatment or 5e6 CD123-CAR NK cells. Leukemia progression was monitored with detection of whole bioluminescence per mouse and analyzed with IVIS software (Perkin Elmer, Waltham, Mass.). Representative images of mice on D28 post-leukemia injection are shown. Line graph is representative of bioluminescence measured per mouse on indicated day post-leukemia injection. BLI displayed as radiance (photons/sec/cm²/sr).

FIG. 15A-D. Demonstration of dual-NK modification. NK cells were transduced with retroviral vectors containing either CLL-ENG paired with the fluorophore mOrange, or a CD123 chimeric receptor. Successful genetic modification was evaluated with FACS analysis and detection of mOrange and/or CAR expression. Unmodified NK cells, B. CLL-ENG NK cells, C. CD123-CAR NK cells, D. CLL-ENG.CD123-CAR NK cells. Circle represents dual modified cells.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a bispecific engager molecule” is a reference to one or more bispecific engager molecules and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “engager” (“ENG”) refers to a molecule that is secreted from a cell and activates immune cells with which it interacts. The engager activates specific immune cells according to the domains present in the engager. Illustrative examples of cells that secrete engagers, but are not limited to, include T-cells, NK cells, NKT cells, CAR T-cells, mesenchymal stem cells (MSCs), neuronal stem cells, hematopoietic stem cells, or a mixture thereof, in some cases.

As used herein, the term “bispecific” refers to any molecule or molecular complex that has two different binding specificities. The molecule or molecular complex may comprise two separate binding domains, each with the same specificity (“homobispecific”) or with specificity for different molecular entities (e.g., antigens) (“heterobispecific”).

As used herein, the term “antigen-recognition domain” refers to a molecular moiety (e.g. part of an engager molecule) that recognizes an antigen. In particular embodiments, antigens can be of any nature including, but not limited to, proteins, carbohydrates, and/or synthetic molecules.

As used herein, the term “activation domain” refers to a molecular moiety (e.g. part of an engager molecule) that interacts with immune cells (e.g., T cell receptor (TCR)) and induces a positive or negative immunomodulatory signal. Illustrative examples of positive immunomodulatory signals include signals that induce cell proliferation, cytokine secretion, or cytolytic activity. Illustrative examples of negative immunomodulatory signals include signals that inhibit cell proliferation, inhibit the secretion of immunosuppressive factors, or induce cell death.

As used herein, the term “native immune cell” refers to an immune cell that naturally occurs in the immune system of a subject. Illustrative examples include, but are not limited to, T-cells, NK cells, NKT cells, B cells, and dendritic cells.

As used herein, the term “engineered immune cell” refers to an immune cell (e.g., T-cell, NK cell, NKT cell, B cell, dendritic cell, etc.) that is genetically modified.

As used herein, the term “co-stimulatory domain” or “co-stimulatory signaling domain” refers to an intracellular signaling domain of a co-stimulatory molecule. In particular aspects, it refers to a domain that provides additional signals to the immune cell in conjunction with an activation domain. Co-stimulatory molecules are cell surface molecules other than antigen receptors or Fc receptors that provide a second signal required for efficient activation and function of T lymphocytes upon binding to antigen. Illustrative examples of such co-stimulatory molecules include CD27, CD28, 4-1BB (CD137), OX40 (CD134), CD30, CD40, ICOS (CD278), LFA-1, CD2, CD7, LIGHT, NKD2C, CD70, CD80, CD86, and CD83.

The term “chimeric antigen receptor” (“CAR”) refers to a recombinant polypeptide construct comprising at least an extracellular antigen-recognition domain, a transmembrane domain and an intracellular signaling domain. Upon binding to their target (e.g., displayed on a cancer cell), CARs typically modify the immune response of the immune cells they are displayed upon.

As used herein, the term “intracellular signaling domain,” when used in reference to a cell surface receptor or a CAR, is a moiety responsible for activation of at least one function of the cell upon which the receptor or CAR is displayed. The term “effector function” refers to a specialized function of a cell. For example, effector function of a T cell includes cytolytic activity or helper activity including the secretion of cytokines. Thus the term “intracellular signaling domain” refers to the portion of a protein which transduces the effector function signal and directs the cell to perform a specialized function. To the extent that a truncated portion or variant of a native intracellular signaling domain is active, such a polypeptide may be used in place of the full native chain, as long as it transduces the effector function signal. The term intracellular signaling domain includes any truncated or variant portion of a polypeptide sequence sufficient to transduce the effector function signal. Examples of intracellular signaling domains include the cytoplasmic sequences of the T cell receptor (TCR) and co-receptors that act in concert to initiate signal transduction following antigen receptor engagement, as well as any derivative or variant of these sequences and any synthetic sequence that has the same functional capability. Cytoplasmic signaling sequences that act in a stimulatory manner comprise signaling motifs which are known as immunoreceptor tyrosine-based activation motifs (ITAMs). Examples of ITAM containing cytoplasmic signaling sequences include those derived from TCR zeta, FcR gamma, FcR beta, CD3 gamma, CD3 delta, CD3 epsilon, CD3 zeta, CD5, CD22, CD79a, CD79b, and CD66d.

As used herein, the term “transmembrane domain,” when used in reference to a cell surface receptor or a CAR, is a moiety that spans the plasma membrane of the cell and is connected to both the intracellular signaling domain and the extracellular antigen-recognition domain. A transmembrane domain may be derived either from a natural or from a synthetic source. Where the source is natural, the domain may be derived from any membrane-bound or transmembrane protein, for example, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, etc. Alternatively the transmembrane domain may be synthetic, in which case it will comprise predominantly hydrophobic residues such as leucine and valine. In some embodiments, a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular signaling domain. A glycine-serine doublet provides a particularly suitable linker.

As used herein, an “immune response” refers to the action of a cell of the immune system (e.g., T lymphocytes, B lymphocytes, natural killer (NK) cells, macrophages, eosinophils, mast cells, dendritic cells, neutrophils, etc.) and soluble macromolecules produced by any of these cells or the liver (e.g., antibodies, cytokines, and complement) that results in selective targeting, binding to, damage to, destruction of, and/or elimination from a subject of invading pathogens, cells or tissues infected with pathogens, or cancerous cells or other abnormal/diseased-associated cells.

As used herein, the term “immunotherapy” refers to the treatment or prevention of a disease or condition by a method comprising inducing, enhancing, suppressing or otherwise modifying an immune response.

As used herein, the term “adoptive cell transfer” (“ACT”) is the transfer of cells into a patient. The cells may have originated from the patient or from another individual or cell line. The cells are most commonly derived from the immune system, with the goal of improving immune functionality or eliciting a desired immune response. In some embodiments, cells are extracted from a subject, genetically modified (e.g., to express a desired construct (e.g., CAR or endanger molecule)), cultured in vitro, and returned to the subject.

As used herein, the term “antibody” refers to a whole antibody molecule or a fragment thereof (e.g., fragments such as scFv, Fab, Fab′, and F(ab′)₂), unless specified otherwise; an antibody may be a polyclonal or monoclonal antibody, a chimeric antibody, a humanized antibody, a human antibody, etc.

A native antibody typically has a tetrameric structure. A tetramer typically comprises two identical pairs of polypeptide chains, each pair having one light chain (in certain embodiments, about 25 kDa) and one heavy chain (in certain embodiments, about 50-70 kDa). In a native antibody, a heavy chain comprises a variable region, V_{H}, and three constant regions, C_{H1}, C_{H2}, and C_{H3}. The V_{H} domain is at the amino-terminus of the heavy chain, and the C_{H3} domain is at the carboxy-terminus. In a native antibody, a light chain comprises a variable region, V_{L}, and a constant region, C_{L}. The variable region of the light chain is at the amino-terminus of the light chain. In a native antibody, the variable regions of each light/heavy chain pair typically form the antigen binding site. The constant regions are typically responsible for effector function.

In a native antibody, the variable regions typically exhibit the same general structure in which relatively conserved framework regions (FRs) are joined by three hypervariable regions, also called complementarity determining regions (CDRs). The CDRs from the two chains of each pair typically are aligned by the framework regions, which may enable binding to a specific epitope. From N-terminus to C-terminus, both light and heavy chain variable regions typically comprise the domains FR1, CDR1, FR2, CDR2, FR3, CDR3 and FR4. The CDRs on the heavy chain are referred to as H1, H2, and H3, while the CDRs on the light chain are referred to as L1, L2, and L3. Typically, CDR3 is the greatest source of molecular diversity within the antigen-binding site. The assignment of amino acids to each domain is typically in accordance with the definitions of Kabat et al. (1991) Sequences of Proteins of Immunological Interest (National Institutes of Health, Publication No. 91-3242, vols. 1-3, Bethesda, Md.); Chothia, C., and Lesk, A. M. (1987) J. Mol. Biol. 196:901-917; or Chothia, C. et al. Nature 342:878-883 (1989). In the present application, the term “CDR” refers to a CDR from either the light or heavy chain, unless otherwise specified.

As used herein, the term “heavy chain” refers to a polypeptide comprising sufficient heavy chain variable region sequence to confer antigen specificity either alone or in combination with a light chain.

As used herein, the term “light chain” refers to a polypeptide comprising sufficient light chain variable region sequence to confer antigen specificity either alone or in combination with a heavy chain.

As used herein, when an antibody or other entity “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸M⁻¹, >10⁹M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion antigen binding region or a variable region. Antibody fragments include, but are not limited to, Fab, Fab′, F(ab′)₂, Fv, scFv, Fd, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis.

For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises additional constant region, extending between the C_{H1} and C_{H2} domains. An interchain disulfide bond can be formed between two heavy chains of a Fab′ fragment to form a “F(ab′)₂” molecule.

An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen.

Other antibody fragments will be understood by skilled artisans.

As used herein, the term “single-chain bispecific antibody construct” refers to a polypeptide construct comprising two antibody-derived binding domains. In some embodiments herein the two antibody-derived binding domains are an antigen-recognition domain and an activation domain. The binding domains may comprise variable regions (or parts thereof) of an antibody, antibody fragment or derivative thereof, capable of specifically binding to/interacting with a target antigen (e.g., CLL-1) or an activation molecule (e.g., human CD3 antigen). In certain embodiments, a part of a variable region comprises at least one CDR (“Complementary determining region”), such as at least a CDR1, CDR2, or CDR3 region. The two domains/regions in the single chain antibody construct are preferably covalently connected to one another as a single chain. Illustrative examples of bispecific single chain molecules are known in the art and are described in WO 99/54440; Mack, J. Immunol. (1997), 158, 3965-3970; Mack, PNAS, (1995), 92, 7021-7025; Kufer, Cancer Immunol. Immunother., (1997), 45, 193-197; Loffler, Blood, (2000), 95, 6, 2098-2103; and Bruhl, J. Immunol., (2001), 166, 2420-2426; incorporated by reference in their entireties.

As used herein, the terms “administration” and “administering” refer to the act of giving a drug, prodrug, or other agent, or therapeutic to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

### DETAILED DESCRIPTION

Provided here are compositions comprising engineered lymphocytes that secrete bispecific engager molecules, thereby activating T cells in the local environment to kill target cells. In particular embodiments, engineered lymphocytes selectively target CLL-1 positive leukemic cells, both directly and through activation of a subject's own T cells, while sparing CLL-1 negative cells, such as myeloid progenitor cells.

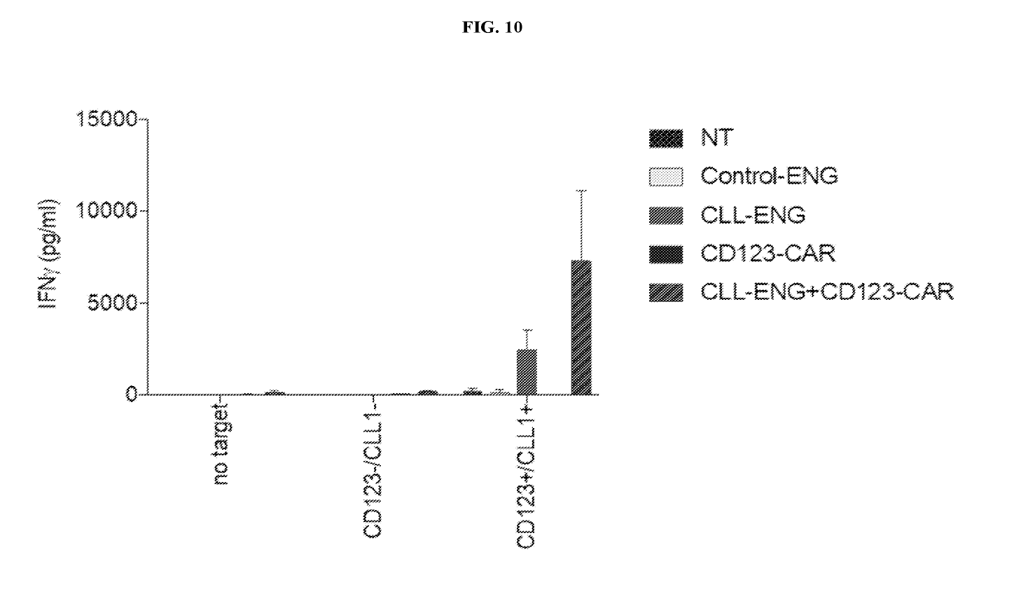

In some embodiments, lymphocytes are engineered to express/display chimeric antigen receptors (CARs) that bind to target cells (e.g., via an antigen-recognition domain), thereby activating the engineered T cells (e.g., via an intracellular signaling domain) toward the target cells (FIG. 10A). In certain embodiments, the engineered lymphocytes display a CAR directed to CLL-1 antigen and/or CD123.

In some embodiments, lymphocytes are engineered to express/secrete bispecific engager molecules that bind to (i) target cells and (ii) engineered and/or native T cells, and thereby activate T cells toward the target cells (FIG. 10B). In certain embodiments, engager molecules target cells displaying a CLL-1 antigen and activate T cells (e.g., engineered and/or native T cells) thereto.

In some embodiments, engineered lymphocytes expressing both a CAR and an engager molecule are provided (FIG. 10C). For example, a lymphocyte may express/display a CAR against a first antigen and express/secrete a bispecific engager molecule against a second antigen and T cell receptor (TCR). The CAR targets the engineered lymphocytes to target cells displaying the first antigen, thereby effecting an immune response by the engineered lymphocyte against the target cell, and localizing the engineered lymphocytes at a treatment site. The engager molecule binds target cells displaying the second antigen (e.g., via its antigen-recognition domain), and also binds T cells (e.g., via its activation domain), thereby effecting an immune response by the engineered lymphocytes as well as native T cells. Localization of the engineered lymphocytes by the CAR, results in release of engager molecules at the treatment cite and concentration of the immune response by the native T cells. The first and second antigens may be the same or different antigens. In some embodiments, the first and second antigens are both displayed on the surface of the target cells.

Some embodiments herein relate to a strategy of adoptive cell transfer of lymphocytes transduced to express a chimeric antigen receptor (CAR), an engager molecule (ENG), or both, for the treatment of cancer (e.g., AML) or other diseases (e.g., infection) in a subject.

In some embodiments, provided herein are compositions comprising engager molecules (e.g., a bispecific engager molecule), wherein the engager molecule comprises (i) a domain that binds to an antigen on an immune cell surface (e.g., native or engineered immune cell surface) and (ii) an domain that binds to a target cell antigen (e.g., an antigen expressed on the surface of tumor cell, cancer cell, or other disease-related cell).

In some embodiments, engager cells (e.g., cells that express and bispecific engager molecules) are provided. In some embodiments, methods are provided comprising administering engager cells to a subject provide therapy (e.g., cancer immunotherapy).

In some embodiments, cells (e.g., immune cells) are genetically modified to express engager molecules comprising at least (i) an antigen-recognition domain and (ii) an activation domain. In some embodiments, engager molecules expressed by engineered cells further comprise one or more accessory domains, such as a cytokine domain, costimulatory domain, a domain for inhibition of negative regulatory molecules of T-cell activation, etc. In some embodiments, an antigen-recognition domain, an activation domain, and/or any accessory domains present in an engager molecule are directly linked or are linked by a linker domain. In some embodiments the linker domain has contained within it a functional moiety, for example, the activation domain of a human cytokine.

In some embodiments, the antigen-recognition domain of an engager molecule binds to one or more molecules present in and/or on target cells or that are secreted by target cells. In particular embodiments, target cells are cancer cells, including at least hematological tumor cells (e.g., hematological malignancies derived from myeloid cell lines). Engager molecules bound to an antigen on a target molecule, are capable of activating immune cells (e.g., engineered or native immune cells) that express/display the molecular determinant recognized by the activation domain of the engager molecule. Engager molecules activate engineered immune cells (e.g., those expressing/secreting engager molecules, those engineered to express other immunotherapeutically-useful agents, etc.) that express/display the molecular determinant recognized by the activation domain. Engager molecules also activate native immune cells (e.g., unmodified immune cells that are native to the subject being treated) that express/display the molecular determinant recognized by the activation domain. Immune cell activation results in a positive or negative signal, depending upon the molecular determinant recognized by the activation domain. Examples of positive signals include signals that induce cell proliferation, cytokine secretion, or cytolytic activity. Examples of negative signals include signals that inhibit-cell proliferation, inhibit the secretion of immunosuppressive factors, or induce cell death. By activating native immune cells, engineered immune cells that secrete engager molecules redirect resident (e.g., naturally endogenous to a specific individual) immune cells to target cells.

In some embodiments, engager molecules comprise a polypeptide chain comprising an antigen-recognition domain and an activation domain. In some embodiments, the antigen-recognition domain and the activation domain are linked directly. In other embodiments, the antigen-recognition domain and the activation domain are connected by a linker peptide. In some embodiments, an engager molecule is a single-chain bispecific antibody construct. In some embodiments, the antigen-recognition domain is a single chain variable fragment that binds a target cell antigen. In some embodiments, the activation domain is a single chain variable fragment that engages the TCR. In some embodiments, both the activation domain and the antigen-recognition domain are scFv's and the individual moieties are arranged and oriented in any suitable manner, for example:

N-V_{H-AD}-L₁-V_{L-AD}-L₂-V_{H-ARD}-L₃-V_{L-ARD}-C;
N-V_{L-AD}-L₁-V_{H-AD}-L₂-V_{H-ARD}-L₃-V_{L-ARD}-C;
N-V_{H-AD}-L₁-V_{L-AD}-L₂-V_{L-ARD}-L₃-V_{H-ARD}-C;
N-V_{L-AD}-L₁-V_{H-AD}-L₂-V_{L-ARD}-L₃-V_{H-ARD}-C;
N-V_{H-ARD}-L₁-V_{L-ARD}-L₂-V_{H-AD}-L₃-V_{L-AD}-C;
N-V_{L-ARD}-L₁-V_{H-ARD}-L₂-V_{H-AD}-L₃-V_{L-AD}-C;
N-V_{H-ARD}-L₁-V_{L-ARD}-L₂-V_{L-AD}-L₃-V_{H-AD}-C;
N-V_{L-ARD}-L₁-V_{H-ARD}-L₂-V_{L-AD}-L₃-V_{H-AD}-C;

wherein N- is the N-terminus; V_{H} is the heavy chain variable region; V_{L} is the light chain variable region; L₁, L₂, and L₃ are linker peptides; —C is the C-terminus; ARD is antigen-recognition domain; and AD is activation domain. In some embodiments, L₁ and L₃ are of appropriate length and sequence to allow the activation scFv and the antigen-binding scFv to each function properly (e.g., bind to TCR and antigen, respectively) individually, and L₂ is or proper length and sequence to allow the activation scFv and antigen-binding scFv to both function (e.g., bind to TCR and antigen) within a single bispecific construct (e.g., engager molecule). In some embodiments, L₁, L₂, and/or L₃ may be absent from the above constructs.

In some embodiments, an engager molecule comprises an antigen-recognition domain that binds to an antigen presented on the surface of a diseased cell or cell that is the source of disease. In particular embodiments, the antigen-recognition domain binds to an antigen presented on a cancer cell or a tumor cell. Any cancer antigen may be targeted by the engager-expressing T-cells or the corresponding engager molecules thereof. In some embodiments, the antigen-recognition domain binds to an antigen presented on cells of hematopoietic and lymphoid malignancies. In some embodiments, the antigen-recognition domain binds to an antigen presented on cells of myeloid malignancies. In some embodiments, the antigen-recognition domain binds to an antigen presented on cells of acute and chronic myelogenous leukemia, myelodysplastic syndromes and myeloproliferative diseases. In certain embodiments, the antigen-recognition domain binds to an antigen presented on cells of myeloid malignancies but not myeloid progenitor cells. In particular embodiments, the antigen-recognition domain binds to C-type lectin domain family 12 member A; a human protein which is encoded by the CLL-1 gene; is also referred to as CLEC12A, CLL1, DCAL-2, MICL, and CD371; and is typically referred to herein as CLL-1. Experiments conducted during development of embodiments herein demonstrate the utility advantageousness of engager molecules comprising CLL-1 antigen-recognition domains and lymphocytes expressing/secreting such engager molecules; however, embodiments herein are not limited to CLL-1 antigen-recognition domains.

In some embodiments, an engager molecule comprises an activation domain that allows the engager molecule to bind to an immunoresponsive cell (e.g., T cell). In some embodiments, the activation domain binds to a surface displayed ligand, antigen, receptor, etc. on the engineered lymphocyte from which it was expressed. In some embodiments, the activation domain binds to a surface displayed ligand, antigen, receptor, etc. on native lymphocytes. In some embodiments, the activation domain binds to native lymphocytes but not engineered lymphocytes. In some embodiments, the activation domain binds to native lymphocytes and engineered lymphocytes. In some embodiments, the activation domain is an antibody or antigen-binding fragment thereof (e.g., scFv). Illustrative examples of activation domains include, but are not limited to antibodies, antigen-binding antibody fragments, ligands, peptides, soluble T-cell receptors, or combinations thereof.

The immune cell to which the engager binds may be an unmodified naturally endogenous (to the recipient individual) immune cell (e.g., native lymphocyte), or it may be a genetically modified immune cell (e.g., engineered lymphocyte). Binding of the engager to the target immune cell (e.g., to the TCR) through the activation domain (e.g., via a CD3 antibody of antibody fragment), thereby activates the target immune cell. When the engager is to target NK cells, the activation domain may comprise of an antibody that recognizes, for example, CD16 (such as NM3E2 antibody), or ligands specific for NKG2D (ULBP2), or NKp30 (B7H6). In specific embodiments, the activation domain comprises ligands, receptors, peptides, etc.

In some embodiments, an engager molecule comprises an activation domain that targets co-stimulatory molecules such as CD27, CD28, CD134, and CD137. In some embodiments, such engager molecules are used in concert with other engager molecules (e.g., engager molecule with activation domains that directly stimulate lymphocytes (e.g., that bind the TCR). For example, T-cells are engineered to express a first engager molecule with a CLL-1-specific antigen recognition domain and a CD3-specific activation domain, and another engager with a CD123-specific antigen recognition domain and a CD28-specific activation domain. T-cells would only be fully activated (e.g., activated by both CD3 and CD28 binding) at tumor sites at which both CLL-1 and CD123 antigens are expressed. In some embodiments, co-stimulatory engager molecules are used in concert with engineered lymphocytes that express/display chimeric antigen receptors. For example, T-cells are engineered to express a chimeric antigen receptor with an CD123-specific antigen recognition domain, and an engager molecule with a CLL-1-specific antigen recognition domain and a CD28-specific activation domain. These engineered T-cells would target any cells displaying a CD123 antigen, but would only be fully activated (at tumor sites at which both CLL-1 and CD123 antigens are expressed. Other combinations of primary (e.g., CD3, TCR, etc.) and co-stimulatory activation domains with engagers, CARs, etc. are within the scope herein.

In some embodiments, in addition to activation, linker, and antigen-recognition domains, an engager molecule comprises one or more additional functional domains (and optionally additional linker domains). Such engager molecules may be trispecific (e.g., capable of binding to three ligands) or multispecifc (e.g., capable of binding to two or more (e.g., 2, 3, 4, 5, 6, 7, 8, or more) ligands) or may be bispecific but further comprise additional functionality (e.g., targeting peptide, therapeutic peptide, luminescence, fluorescence, etc.). Additional functional domains may comprise a cytokine, costimulatory domain, and/or domain for inhibition of negative regulatory molecules of T-cell activation.

As addressed above, in some embodiments, engager molecules comprise one or more linker domains (e.g., between heavy and light variable chains, between activation and antigen-recognition domains, etc.). Linkers that facilitate the formation and activity of scFv constructs (e.g., L₁- and L₃-type linkers, linkers between heavy and light variable chains) are well understood in the art. In some embodiments, such linkers are about 5 to 50 amino acids in length (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or ranges therebetween (e.g., 10-25)). In some embodiments, linkers are glycine rich (e.g., >20%, >30%, >40%, >50%) to impart flexibility to the linker. In some embodiments, linkers are serine and/or threonine rich (e.g., >20%, >30%, >40%, >50%) to impart solubility to the linker. In some embodiments, linkers are of any suitable length and sequence so as to allow the formation of active activation and antigen-recognition domains. In some embodiments, linkers between the activation and antigen-recognition domains (e.g., L₂-type linkers) comprise similar characteristics (e.g., to L₁- and L₃-type linkers). In some embodiments, linkers between the activation and antigen-recognition domains (e.g., L₂-type linkers) are of any suitable length and sequence so as to allow the formation of active activation and antigen-recognition domains, without adverse interaction occurring between the domains.

In some embodiments, provided herein are chimeric antigen receptors (CARs) and lymphocytes that are engineered to express/display a desired CAR (e.g., engineered to express a CLL1-targetted CAR alone, engineered to express a CAR and an engager molecule, etc.). In some embodiments, a cell is engineered to stably express an antibody binding domain on its surface, conferring novel antigen specificity (e.g., that is MHC independent). In some instances, a cell is engineered to express a CAR that combines an antigen recognition domain of a specific antibody with an intracellular activation domain (e.g., CD3-zeta chain or FcyRI protein) into a single chimeric protein.

In some embodiments, a CAR comprises an extracellular domain having an antigen recognition domain, a transmembrane domain, and a cytoplasmic domain having an intracellular signaling domain. In some embodiments, the transmembrane domain that naturally is associated with one of the domains in the CAR is used. In another embodiment, the transmembrane domain is selected or modified by amino acid substitution to avoid binding of such domains to the transmembrane domains of the same or different surface membrane proteins to minimize interactions with other members of the receptor complex. In some embodiments, the transmembrane domain is the CD8 hinge domain.

With respect to the cytoplasmic domain, in some embodiments a CAR comprises the CD28 and/or 4-IBB signaling domain by itself or is combined with any other desired cytoplasmic domain(s) useful in the context of the CAR. In some embodiments, the cytoplasmic domain of the CAR comprises the signaling domain of CD3-zeta. For example, the cytoplasmic domain of the CAR includes but is not limited to CD3-zeta, 4-IBB and CD28 signaling modules and combinations thereof.

Chimeric antigen receptors provided herein comprise an extracellular and intracellular domain. The extracellular domain comprises an antigen recognition domain. In some embodiments, the intracellular domain or the cytoplasmic domain comprises, a costimulatory signaling region and a zeta chain portion. The costimulatory signaling region refers to a portion of the CAR comprising the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the CAR, or between the cytoplasmic domain and the transmembrane domain of the CAR, there may be incorporated a linker domain. A linker domain of a CAR is an oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

In some embodiments, a CAR comprises an antigen recognition domain. The choice of antigen recognition domain depends upon the type and number of ligands that define the surface of a target cell. For example, the antigen recognition domain may be chosen to recognize a ligand that acts as a cell surface marker on target cells associated with a particular disease state. Thus examples of cell surface markers that may act as ligands for the antigen moiety domain in the CAR of include those associated with viral, bacterial and parasitic infections, autoimmune disease and cancer cells.

In some embodiments, a CAR targets a tumor antigen of interest by displaying an antigen recognition domain that specifically binds to an antigen on a cancer cell. Cancer cell antigens are proteins that are produced by cancer cells that elicit an immune response, particularly T-cell mediated immune responses. The selection of the antigen recognition domain depends on the particular type of cancer to be treated. Cancer antigens are well known in the art. Examples of cancer antigens that may be targeted by CARs in embodiments herein include CLL-1 and CD123 antigens. In some embodiments, the cancer antigen comprises one or more antigenic cancer epitopes associated with a malignant tumor, metastatic tumor, leukemia (e.g., AML), etc.

The type of cancer antigen targeted herein (e.g., by CAR and/or by bispecific engager) may also be a cancer-specific antigen (CSA) (or tumor-specific antigen (TSA)) or a cancer-associated-antigen (CAA) (or tumor-associated-antigen (TAA)). A CSA is unique to cancer cells and does not occur on other cells in the body (e.g., healthy native cells). A CAA is not unique to a tumor cell and instead is also expressed on normal cells under conditions.

Depending on the desired antigen to be targeted, a CAR is engineered to include the appropriate antigen recognition domain that is specific to the desired antigen target. For example, if CD123 or CLL-1 is the desired antigen that is to be targeted, an antibody (or fragment thereof (e.g., scFv)) for CD123 or CLL-1 is used as the antigen recognition domain for incorporation into the CAR.

In some embodiments, the antigen recognition domain of a CAR targets CD123. In some embodiments, the antigen recognition domain in the CAR of the invention is anti-CD123 scFV.

In some embodiments, the antigen recognition domain of a CAR targets CLL-1. In some embodiments, the antigen recognition domain in the CAR of the invention is anti-CLL-1 scFV.

With respect to the transmembrane domain, in some embodiments, CARs are designed to comprise a transmembrane domain that is fused to the extracellular and intracellular domains of the CAR. In some embodiments, a transmembrane domain is a sequence that is naturally associated with one of the other domains in the CAR. In some embodiments, the transmembrane domain is selected or modified by amino acid substitution to avoid interactions with other CAR domains or cell surface components.

In some embodiments, a transmembrane domain is from either a natural or a synthetic source. Where the source is natural, the domain may be derived from any membrane-bound or transmembrane protein. Transmembrane regions of particular use include at least the transmembrane region(s) of known transmembrane proteins, including, but not limited to: the alpha, beta or zeta chain of the T-cell receptor, CD28, CD3 epsilon, CD45, CD4, CD5, CDS, CD9, CD 16, CD22, CD33, CD37, CD64, CD80, CD86, CD 134, CD137, CD 154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic domain of the CAR. A glycine-serine doublet provides a particularly suitable linker.

In some embodiments, the transmembrane domain of the CAR comprises a CD8 hinge domain.

The cytoplasmic domain (a.k.a. intracellular signaling domain, activation domain, etc.) of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While the entire intracellular signaling domain of a known protein or protein complex may be employed in certain embodiments, in other embodiments it is not necessary to use the entire chain. To the extent that a truncated portion of a known intracellular signaling domain is used, such truncated portion may be used in place of the intact chain as long as it transduces the effector function signal. Examples of intracellular signaling domains for use in the CARs herein include the cytoplasmic sequences of the T cell receptor (TCR) and co-receptors that act in concert to initiate signal transduction following antigen-receptor engagement, as well as any derivative or variant of these sequences and any synthetic sequence that has the same functional capability.

Signals generated through the TCR alone are insufficient for full activation of the T cell; a secondary or co-stimulatory signal is also required for full activation. Thus, in some embodiments, T cell activation is mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences). Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of 1TAM containing primary cytoplasmic signaling sequences that are of particular use in the invention include those derived from TCR zeta, FcR gamma, FcR beta, CD3 gamma, CD3 delta, CD3 epsilon, CDS, CD22, CD79a, CD79b, and CD66d. It is particularly preferred that cytoplasmic signaling molecule in the CAR of the invention comprises a cytoplasmic signaling sequence derived from CD3 zeta.

In some embodiments, the cytoplasmic domain of the CAR comprises a primary signaling sequence (e.g., CD3-zeta signaling domain) by itself or combined with any other desired cytoplasmic domain(s) useful in the context of the CAR. For example, the cytoplasmic domain of the CAR may comprise a primary signaling sequence and a costimulatory signaling region. The costimuiatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1 BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like.

The cytoplasmic signaling sequences within the cytoplasmic signaling portion of the CAR may be linked to each other in a random or specified order. Optionally, a short oligopeptide linker (e.g., between 2 and 25 amino acids in length) forms the linkage. A glycine-serine doublet provides a particularly suitable linker.

In some embodiments, provided herein are lymphocytes engineered to express one or more chimeric antigen receptors (CARs) and/or engager molecules. Engineered cells may be generated by any suitable method in the art. In specific embodiments, the engineered lymphocytes are generated by viral transduction of lymphocytes, (e.g., T-cell, NK cell, NKT cell, B cell, dendritic cell, etc.). In some embodiments, lymphocytes are engineered to express/display one or more CARs (e.g., targeting CD123, CLL-1, and/or other antigens). In some embodiments, lymphocytes are engineered to express/secrete one or more engager molecules (e.g., targeting CD123, CLL-1, and/or other antigens). In some embodiments, lymphocytes are engineered to express/display one or more CARs (e.g., targeting CD123, CLL-1, and/or other antigens) and to express/secrete one or more engager molecules (e.g., targeting CD123, CLL-1, and/or other antigens).

Provided herein are nucleic acids and nucleic acid sequences encoding bispecific engager molecules and CARs as described above and cells harboring such nucleic acids. In some embodiments, nucleic acid molecules are recombinant nucleic acid molecules. In some embodiments, nucleic acid molecules are synthetic. Nucleic acids encoding bispecific engager molecules and CARs may comprise DNA, RNA, PNA (peptide nucleic acid), and hybrids thereof.

In some embodiments, a nucleic acid encoding a bispecific engager molecule and/or CAR comprises one or more regulatory sequences. For example, promoters, transcriptional enhancers and/or sequences that allow for induced expression of the polynucleotide of the disclosure may be employed. In some embodiments, nucleic acid molecules are transcribed by an appropriate vector comprising a chimeric gene that allows for the transcription of the nucleic acid molecule in the cell.

In some embodiments, a nucleic acid molecule is a recombinantly-produced chimeric nucleic acid molecule comprising any of the aforementioned nucleic acid molecules either alone or in combination. In some embodiments, the nucleic acid molecule is part of a vector.

In some embodiments, provided herein are vectors comprising the nucleic acid molecule described herein (e.g., encoding CARs and/or engager molecules). Many suitable vectors are known to those skilled in molecular biology, the choice of which would depend on the function desired and include plasmids, cosmids, viruses, bacteriophages and other vectors used conventionally in genetic engineering. Methods that are well known to those skilled in the art can be used to construct various plasmids and vectors; see, for example, the techniques described in Sambrook et al. (1989) and Ausubel, Current Protocols in Molecular Biology, Green Publishing Associates and Wiley Interscience, N.Y. (1989), (1994); incorporated by reference in its entirety. Alternatively, the polynucleotides and vectors of the disclosure are reconstituted into liposomes for delivery to target cells. A cloning vector may be used to isolate individual sequences of DNA. Relevant sequences can be transferred into expression vectors where expression of a particular polypeptide is required. Typical cloning vectors include pBluescript SK, pGEM, pUC9, pBR322 and pGBT9. Typical expression vectors include pTRE, pCAL-n-EK, pESP-1, pOP13CAT.

In some embodiments, a vector comprises a nucleic acid sequence that is a regulatory sequence operably linked to the nucleic acid sequence encoding a CAR and/or engager molecule described herein. Such regulatory sequences (control elements) are known to the artisan and may include a promoter, a splice cassette, translation initiation codon, and insertion site for introducing an insert into the vector. In specific embodiments, the nucleic acid molecule is operatively linked to said expression control sequences allowing expression in eukaryotic or prokaryotic cells.

In some embodiments, the vector is a viral vector, such as a lentiviral vector or adenovirus associate vector.

In some embodiments, nucleic acids and/or vectors are used in a cell to express encoded polypeptides (e.g., CARS, engager molecules, etc.) in the cells. The nucleic acid molecules or vectors containing the DNA sequence(s) encoding any of the CAR and/or engager constructs herein are introduced into the cells that in turn produce the polypeptide(s). The recited nucleic acid molecules and vectors may be designed for direct introduction or for introduction via liposomes, or viral vectors (e.g., adenoviral, retroviral) into a cell. In certain embodiments, the cells are T-cells, CAR T-cells, NK cells, NKT-cells, MSCs, etc.

In accordance with the above, provided herein are methods to derive vectors, particularly plasmids, cosmids, viruses and bacteriophages used conventionally in genetic engineering that comprise a nucleic acid molecule encoding a polypeptide sequence (e.g., a CAR and/or engager) described herein. In some embodiments, a vector is an expression vector and/or a gene transfer or targeting vector. Expression vectors derived from viruses such as retroviruses, vaccinia virus, adeno-associated virus, herpes viruses, or bovine papilloma virus, may be used for delivery of polynucleotides and/or vectors into targeted cell populations. Methods which are well known to those skilled in the art can be used to construct recombinant vectors. Vectors are transferred into the host cells by well-known methods, which vary depending on the type of cellular host.

In some embodiments, provided herein are cells comprising a host cell transformed or transfected with a vector defined herein above (e.g., encoding a engager or CAR described herein). The host cell may be produced by introducing at least one of the above described vectors or at least one of the above described nucleic acid molecules into the host cell. The presence of the at least one vector or at least one nucleic acid molecule in the host may mediate the expression of a gene encoding the above described CAR and/or engager. The nucleic acid molecule or vector that is introduced in the host cell may either integrate into the genome of the host or it may be maintained extrachromosomally.

In some embodiments, provided herein are methods comprising culturing a host cell defined herein above under conditions allowing the introduction of the nucleic acid and/or vector. In some embodiments, provided herein are methods comprising culturing a host cell defined herein above under conditions allowing expression of a construct (e.g., comprising a CAR and/or engager). In particular embodiments, the cultured cells (e.g., expressing/displaying a CAR and/or expressing/secreting an engager molecule) are provided to a subject (e.g., from which the original cells were obtained, a second subject, etc.). Conditions for the culturing of cells harboring an expression construct are known in the art.

In some embodiments, lymphocytes for engineering according to embodiments herein are from any suitable source. For example, a source of lymphocytes is a subject (e.g., the subject to be treated, a healthy subject, etc.). Lymphocytes can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, a specific type of lymphocyte (e.g., T cell, NK cell, B cell, etc.) desired for an embodiment described herein is obtained by appropriate methods. In some embodiments, lymphocytes expressing a particular marker are obtained by known methods (e.g., cell sorting). In some embodiments, cells are cultured following isolation. In some embodiments, cells are engineered using methods described herein.

In various embodiments herein, engager molecules, CARs, nucleic acid sequences, vectors, host cells, etc. as contemplated herein and/or pharmaceutical compositions comprising the same are used for the prevention, treatment or amelioration of a cancerous disease, such as, for example AML.

In some embodiments, compositions herein (e.g., CAR-cells, ENG-cells, nucleic acid molecules and vectors, etc.) are administered either alone or in any combination using standard delivery systems and methods, and in at least some aspects, together with a pharmaceutically acceptable carrier or excipient. In the case of nucleic acid molecules or vectors, they may be stably integrated into the genome of the subject.

In some embodiments, methods and compositions are provided relating to the prevention, treatment or amelioration of a cancer comprising the step of administering to a subject in the need thereof an effective amount of cells harboring an engager molecule, CAR, a nucleic acid sequence, a vector, as contemplated herein and/or produced by a process as contemplated herein. When cells are administered, the engineered cells are either administered to a site of treatment or may localize at a site of treatment (e.g., cell type, tissue type, etc.).

In some embodiments, indications for administration of the composition(s) herein are cancerous diseases. Examples of hematological (or hematogenous) cancers that are treated/prevented in embodiments herein include leukemias, including acute leukemias (such as acute lymphocytic leukemia, acute myelocytic leukemia, acute myelogenous leukemia and myeloblasts, promyeiocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukemia and myelodysplasia. Examples of solid tumors that are treated/prevented in embodiments herein include, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pineaioma, hemangioblastoma, acoustic neuroma, oligodendroglioma, menangioma, neuroblastoma, retinoblastoma and brain metastases).

The disclosure further encompasses co-administration protocols with other compounds, e.g. bispecific antibody constructs, targeted toxins or other blocking or functional antibodies or compounds, which act via immune cells. The clinical regimen for co-administration may encompass co-administration at the same time, before or after the administration of the other component. Particular combination therapies include chemotherapy, radiation, surgery, hormone therapy, or other types of immunotherapy. Many chemotherapeutics are presently known in the art and can be used in combination with the compounds of the invention. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

In some embodiments, the engineered lymphocytes described herein are co-administered with one or more chemotherapeutics. Chemotherapies for use with the engineered lymphocytes described herein include all classes of chemotherapeutic agents, such as, alkylating agents, antimetabalites, plant alkaloids, antibiotics, hormonal agents, and miscellaneous anticancer drugs. Specific agents include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabine, fuldarabine, navelbine, farnesyl-protein tansferase inhibitors, transplatinum, 5-fluorouracil, vincristin, and vinblastin, or any analog or derivative variant of the foregoing and also combinations thereof. In some embodiments, chemotherapy is employed before, during and/or after administration of engineered lymphocytes.

In some embodiments, the engineered lymphocytes described herein are co-administered with radiotherapy, methods of which are understood in the field. In some embodiments, radiotherapy is employed before, during and/or after administration of engineered lymphocytes.

In some embodiments, the engineered lymphocytes described herein are co-administered with non-immune based targeted therapies, such as, agents that inhibit signaling pathways such WNT, p53, and/or RB-signaling pathways. Other examples include agents that inhibit tyrosine kinases, BRAF, STAT3, c-met, regulate gene expression, induce cell death or block blood vessel formation. Examples of specific agents include imatinib mesylate, dasatinib, nilotinib, bosutinib, lapatinib, gefinitib, erlotinib, tensirolimus, everolimus, vemurafenib, crizotinib, vorinostat, romidepsin, bexarotene, alitrionin, tretionin, bortezomib, carfilzomib, pralatrexate, sorafenib, sunitinib, pazopanib, regorafenib, or cabozantinib. In some embodiments, non-immune based targeted therapy is employed before, during and/or after administration of engineered lymphocytes.

In some embodiments, the engineered lymphocytes described herein are co-administered with an immunotherapy. Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect-cell killing. The antibody may also prevent cancer immunoevasion or immunosuppression. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T-cells, NKT cells, and NK cells. In some embodiments, immunotherapy is employed before, during and/or after administration of engineered lymphocytes. In some embodiments, engineered lymphocytes are co-administered with an immune checkpoint inhibitor (e.g., anti-PD1, anti-PDL1, anti-CTLA-4, etc.).

In some embodiments, the engineered lymphocytes described herein are co-administered with a gene therapy in which a therapeutic polynucleotide is administered before, after, or at the same time as the engineered lymphocytes described herein. A variety of expression products are encompassed, including inducers of cellular proliferation, inhibitors of cellular proliferation, or regulators of programmed cell death.

In some embodiments, the engineered lymphocytes described herein are administered before, during, and/or after surgery. Surgeries include resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that embodiments herein may be used in conjunction with removal of superficial cancers, precancers, or incidental amounts of normal tissue.

In some embodiments, the engineered lymphocytes described herein are co-administered with other agents to improve the therapeutic efficacy of treatment.

In some embodiments, engineered lymphocytes described herein are provided as part of a kit or system along with one or more additional components, such as instructions, devices for administration, additional therapeutic agents, diagnostic agents, research agents, etc.

### EXPERIMENTAL

### Example 1

### CLL1-ENG

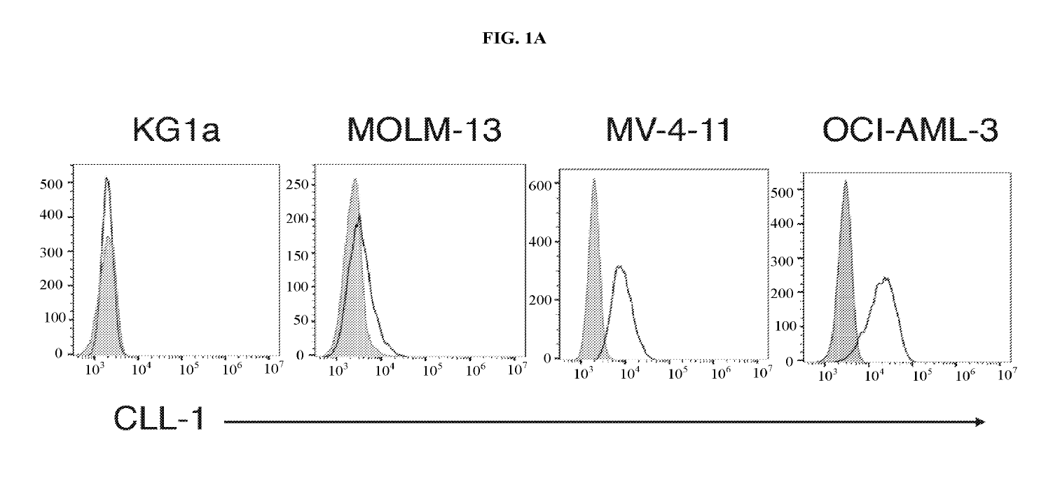

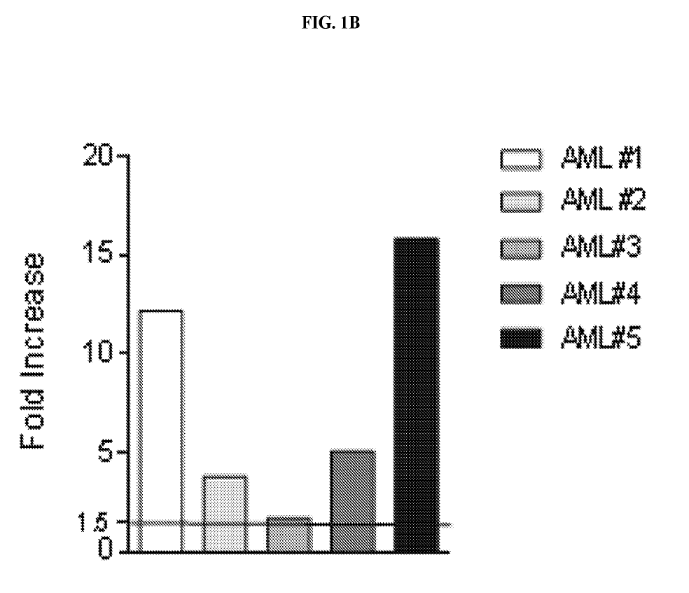

Experiments conducted during development of embodiments herein demonstrate that CLL-1 is expressed on a range of human myeloid leukemia cell lines and primary acute myeloid leukemia samples. Flow cytometry analysis of established KG1a, MOLM-13, MV-4-11, and OCIAML-3, human myeloid leukemia cell lines using anti-human CLL-1-FITC (FIG. 1A) and analysis of primary human acute myeloid leukemia samples (FIG. 1B) demonstrate the presence of CLL-1 on the surface of human myeloid leukemia cells.

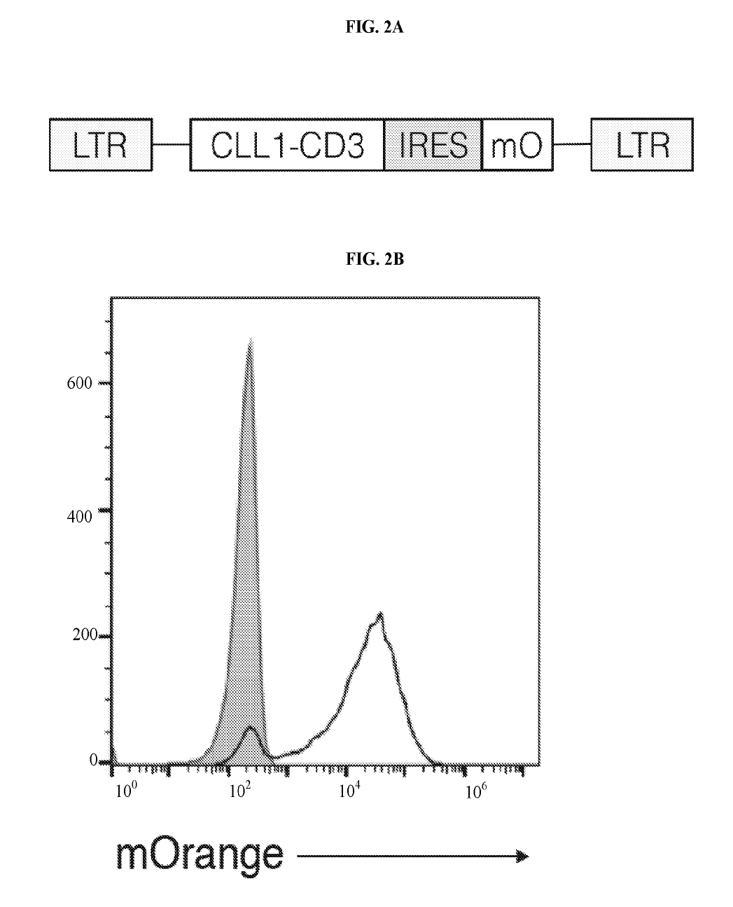

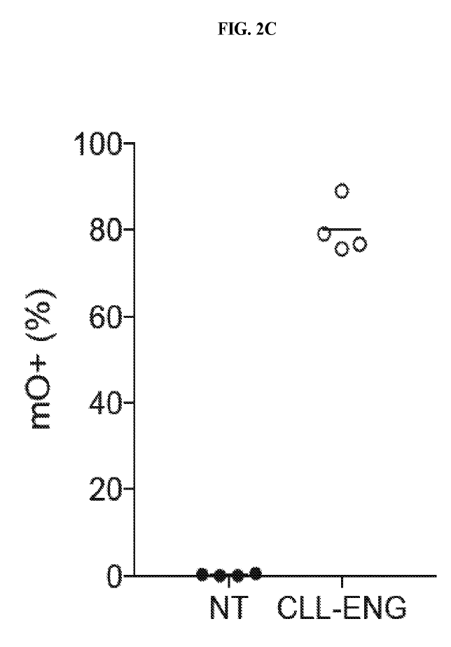

Experiments conducted during development of embodiments herein demonstrate that human T cells can be transduced to express bispecific engager molecules with an antigen-recongition domain specific for CLL-1 and an activation domain specific for CD3c, an integral component of the T-cell receptor complex (CLL1-ENG; FIG. 2A). Flow cytometry analysis demonstrates the successful transduction of human T cells with the CLL1-ENG retroviral vector.

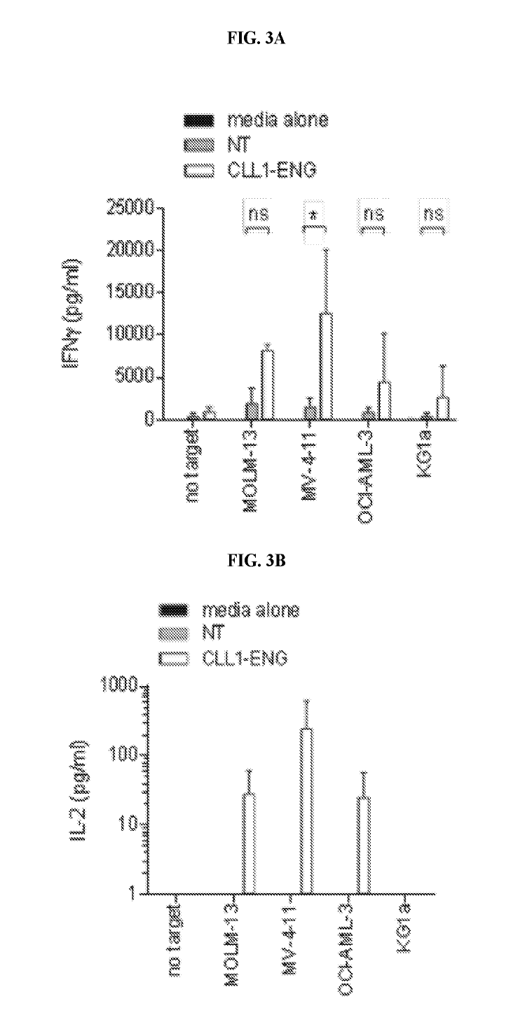

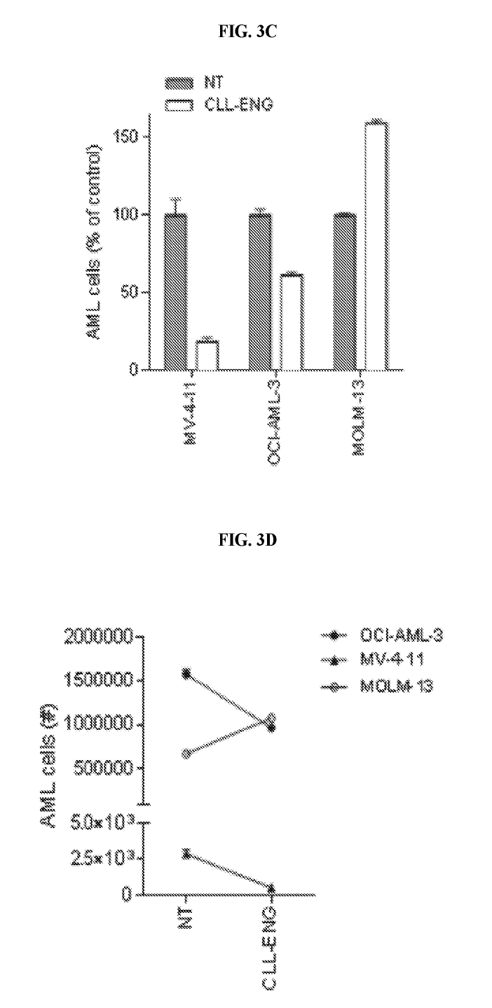

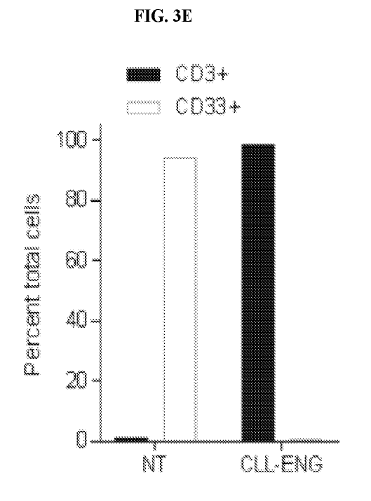

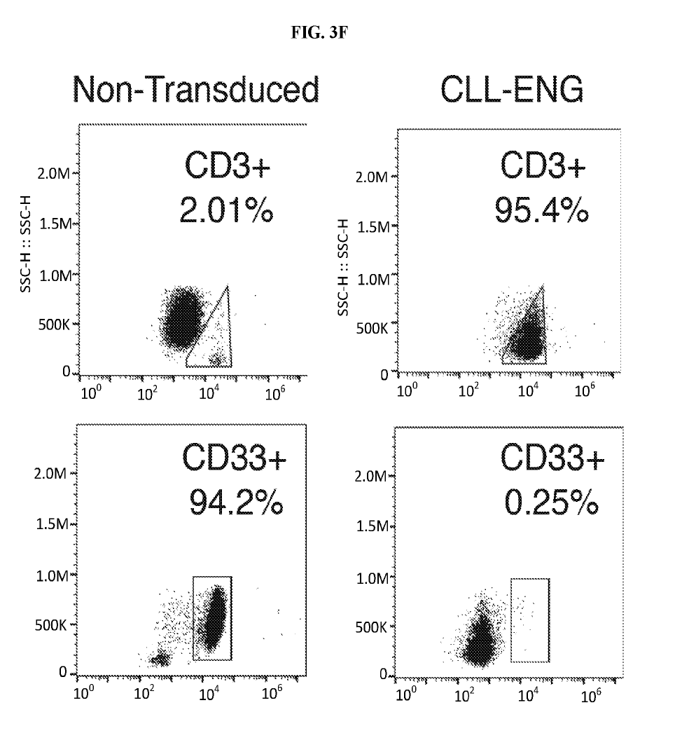

Experiments conducted during development of embodiments herein to demonstrate of CLL1-ENG T cell activation and functional activity versus CLL-1-positive target cells (FIG. 3). Co-culture of CLL1-ENG T cells and CLL1+ targets cells results in T cell activation. Measurement of IFNγ and IL-2, cytokines secreted by activated T cells, in the cell culture supernatant of selectively activated CLL1-ENG T cells proves specific T-cell activation (FIG. 3A, B). Co-culture of CLL1-ENG T cells and the CLL-1+ target cells MV-4-11 and OCI-AML-3 results in death of target cells. Co-culture of CLL-1 ENG T cells and the CLL-1 negative MOLM-13 AML cell line does not result in MOLM-13 cell death. This is demonstrated by measurement of cell viability following CLL1-ENG T cell and AML cell co-culture with bioluminescence measurement in AML lines stably transduced to express firefly Luciferase and treated with D-Luciferin (FIG. 3C), as well as FACS analysis following co-culture for the AML surface antigen CD33 and the T cell antigen CD3 (FIG. 3D,E).

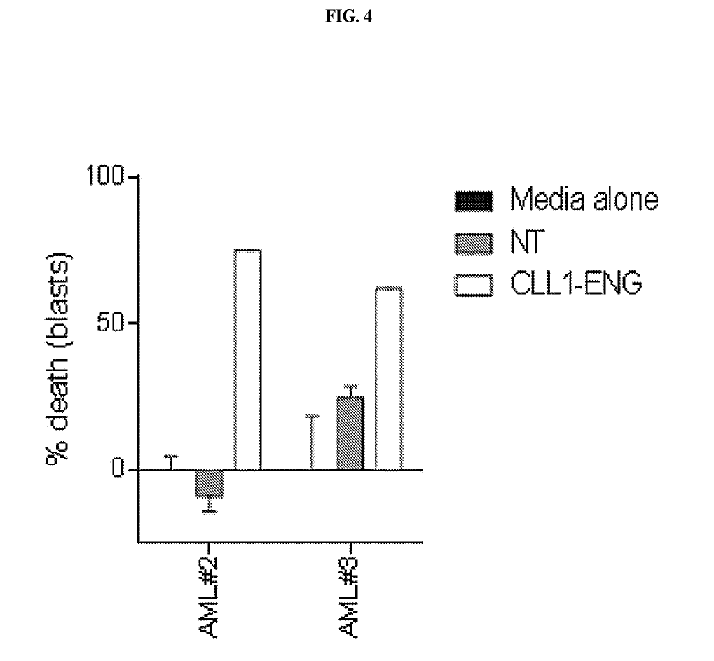

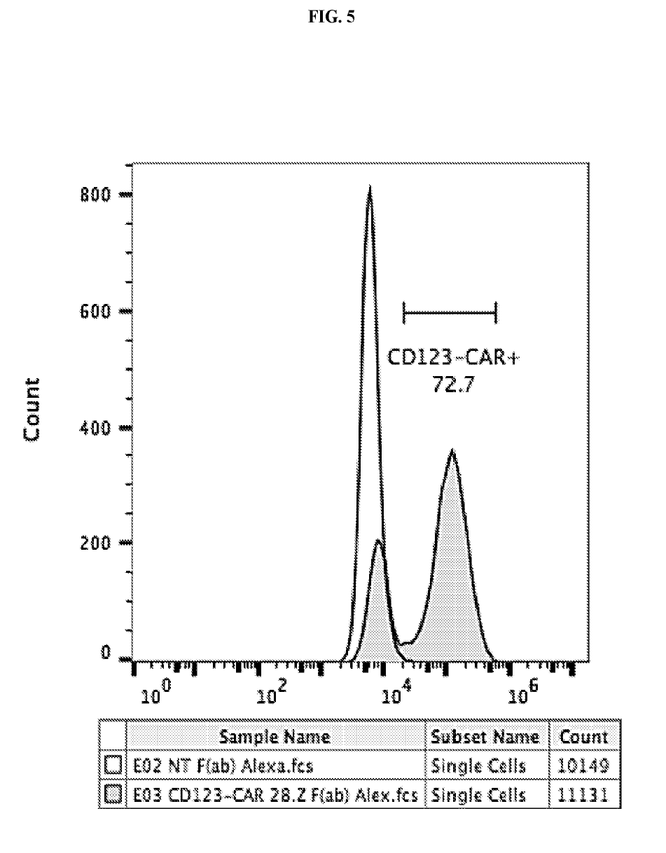

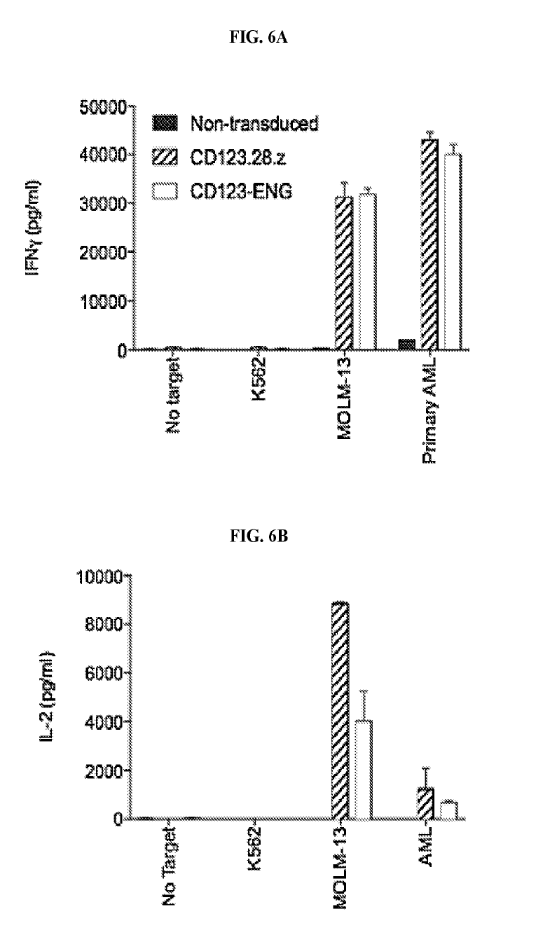

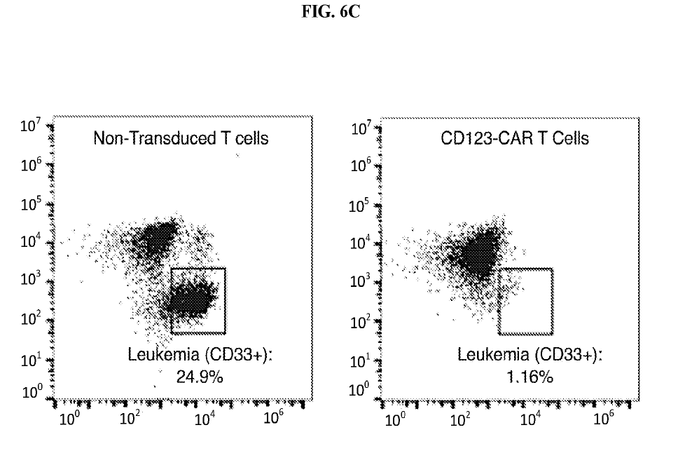

Experiments conducted during development of embodiments herein to demonstrate CLL1-ENG activation of autologous primary T cells versus acute myeloid leukemia (FIG. 4). Primary peripheral blood samples containing both AML cells and T cells were plated below a transwell. The transwell allows for the transmission of small molecules, such as the CLL1-ENG, but not cells. When CLL-1 ENG T cells that secrete CLL1 ENG are plated in the top transwell, the ENG molecule travels through the membrane to activate primary autologous T cells to kill AML blasts present in the culture. Media alone or non-transduced T cells plated in the upper chamber do not activate autologous T cells to kill AML blasts.

### Example 2

### CD123-CAR

Experiments conducted during development of embodiments herein CD123-CAR expression. FACS analysis performed following retroviral transduction of activated T-cells with retroviral construct containing CD123 scFv, CD28 co-stimulatory region, and CD3c intracellular signaling domain. As the retroviral vector construct contains the coding sequence for the fluorescent protein mOrange downstream of an IRES, FACS analysis for mOrange expression proves high efficiency transduction of T cells.

Experiments conducted during development of embodiments herein demonstrating CD123-CAR T cell activation and cytotoxicity versus CD123+ target cells. Co-culture of CD123-CAR T cells and CD123+ targets cells (MOLM-13, primary AML) results in T cell activation. Measurement of IFNγ and IL-2, cytokines secreted by activated T cells, in the cell culture supernatant of selectively activated CD123-CAR T cells proves specific T-cell activation. CD123-CAR T cells in culture with CD123-negative targets (no target, K562) does not result in cytokine secretion. CD123-CAR T cells, when cultured with CD123_, CD33+MOLM-13 AML cells efficiently kill target cells as measured by FACS analysis for CD33+ cells. Non-transduced T cells exhibit no cytotoxicity vs. target cells.

### Example 3

### CD123(CAR)/CLL-1(ENG) T Cells

### Generation and Functional Characterization of CD123 and CLL-1 Targeted T Cells

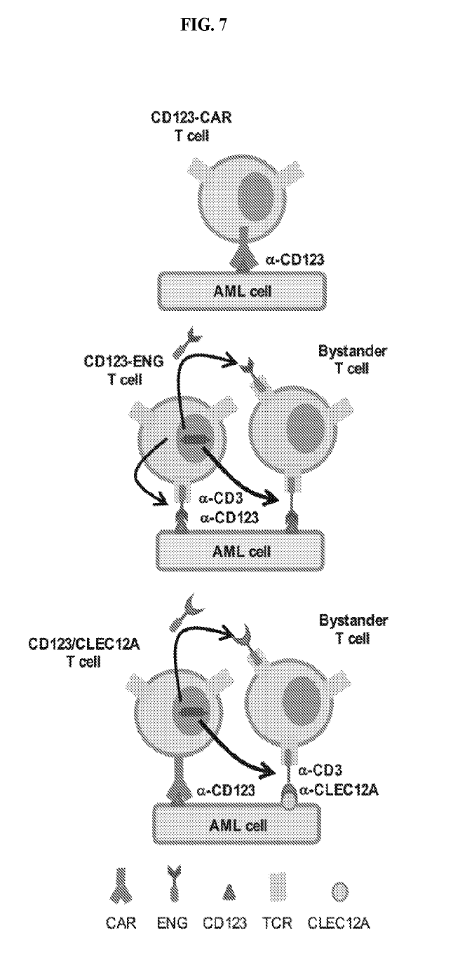

Expression of CD123 and CLL-1 were measured on AML cell lines and primary myeloid blasts with flow cytometric analysis (FIG. 7A, B, C). A codon-optimized CD123- or CLL-1-ENG containing of a CD123- or CLL1-specific scFv (Du et al. J Immunother 30, 607-613 (2007).; Abo & Korver, U.S. Pat. No. 8,536,310 (2013).; incorporated by reference in their entireties), a short serine-glycine linker, and an scFv specific for CD3E was subcloned into a retroviral vector. Transduction of normal donors was both efficient and robust.

The proven effective CD123-specific scFv are used to generate CD123-CART cells and Experiments are conducted using CD123-CAR T against CD123+ and CD123-target cells. CD123-CAR is then co-expressed with CLL-1-ENG to determine the ability of these cells to (i) produce cytokines, (ii) proliferate, (iii) kill AML tumor cells in vitro, and (iv) persist with ongoing anti-tumor activity in our established xenograft models in vivo. Standard in vitro immunological assays and MOLM-13.ffLuc NSG (NOD-scid IL2Rg^{null}) leukemia model for in vivo studies are used for evaluations, as well as primary human leukemia xenografts established with patient samples (HUM000105312). An OCI-AML-3.ffLuc cell line has been prepared to facilitate comparative recognition of CLL-1+(OCI-AML-3) and CLL-1-(MOLM-13) leukemias. Experiments will also evaluate downstream TCR signaling to differentiate this from signaling through the chimeric receptor. Experiments include intracellular cytokine staining (mOrange+, transduced; mOrange-, bystander T cell) as well as Western blot analysis. Physically separation of ENG-T from CAR-T using a transwell format is achievable, and cells can be sorted prior to analysis. The effect of ENG and CAR activation is separated to compare and contrast the contribution of each to AML pathogenic control.

### Example 4

### Demonstration of CLL-ENG Activity In Vivo

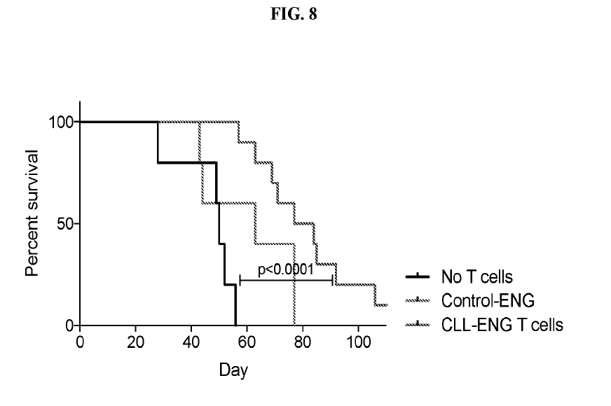

Experiments were conducted during development of embodiments herein to demonstrate CLL-ENG activity in vivo (FIG. 8). Mice were administered leukemia cells and then were given either no treatment, treated with control ENG-T cells, or treated with CLL-ENG T cells. Leukemia progression was monitored. The results demonstrate that the administration of the CLL-ENG T cells prolonged survival compared to both of the controls.

### Example 5

### Co-Expression of CLL-ENG and CD123-CAR in T Cells

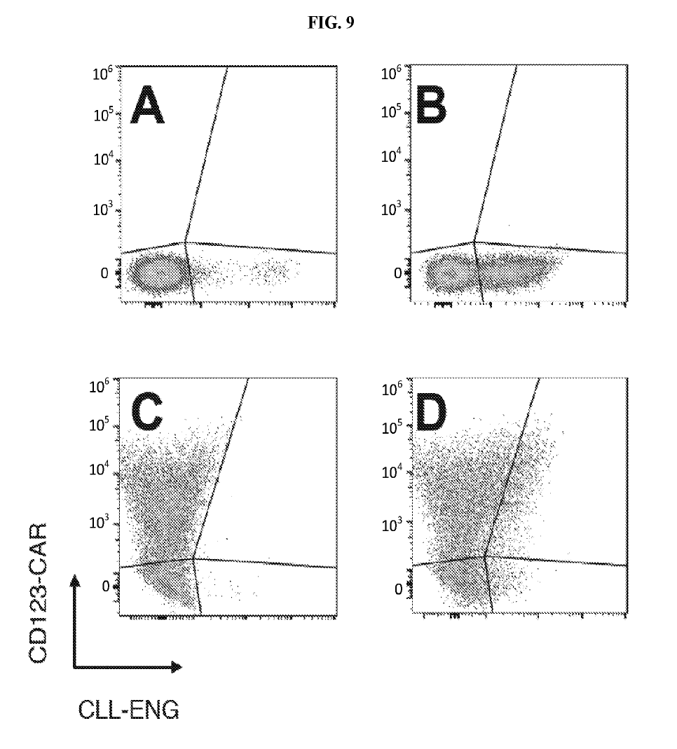

Experiments were conducted during development of embodiments herein to demonstrate co-expression of CLL-ENG and CD123-CAR in T cells. T cells were transduced with retroviral vectors containing either CLL-ENG paired with the flurophore mOrange, or a CD123 chimeric receptor paired with the fluorophore ZsGreen. Successful genetic modification was evaluated with FACS analysis and detection of mOrange and/or ZsGreen. Dual modified cells were detected (FIG. 9D; circle).

### Example 6

### Enhanced Activation with Dual T Cell Modification

Experiments were conducted during development of embodiments herein to demonstrate enhancement of activation by dual T cell modification. Target cells (CD123-negative/CLL1-negative or CD123-positive/CLL1-positive) were plated in co-culture with unmodified, control-ENG, CLL-ENG, CD123-CAR, or CLL-ENG/CD123-CAR T cells. After 24 hours of culture, supernatant was harvested and assessed for the presence of Interferon-gamma as a marker of T cell activation. Significant enhancement of T cell activation is observed by the dual-modified cells (FIG. 10).

### Example 7

### Lack of Toxicity to Stem and Progenitor Hematopoietic Cells by CLL1-Targeting

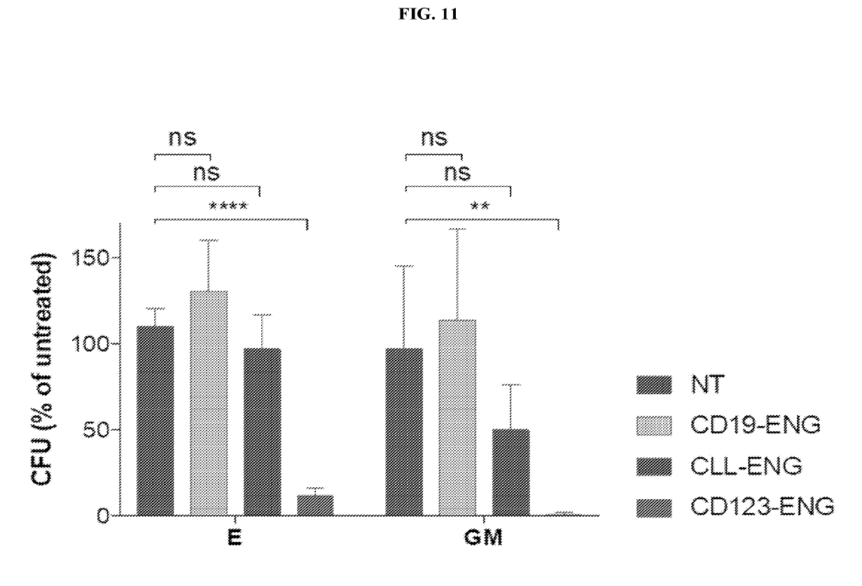

Experiments were conducted during development of embodiments herein to assess toxicity to stem and progenitor hematopoietic cells by CLL1-targeting (FIG. 11). Bone marrow mononuclear cells (BMMC), isolated from healthy donor bone marrow via a density gradient, were plated and incubated with T cells (unmodified (NT), CD19-ENG, CLL-ENG, and CD123-ENG). After incubation, cells were plated and incubated in semisolid media containing growth factors. The resulting colonies were counted manually, identified as CFU-E or CFU-GM, and the count was then normalized to that of identical conditions without T cells present.

### Example 8

### CAR-NK Modification

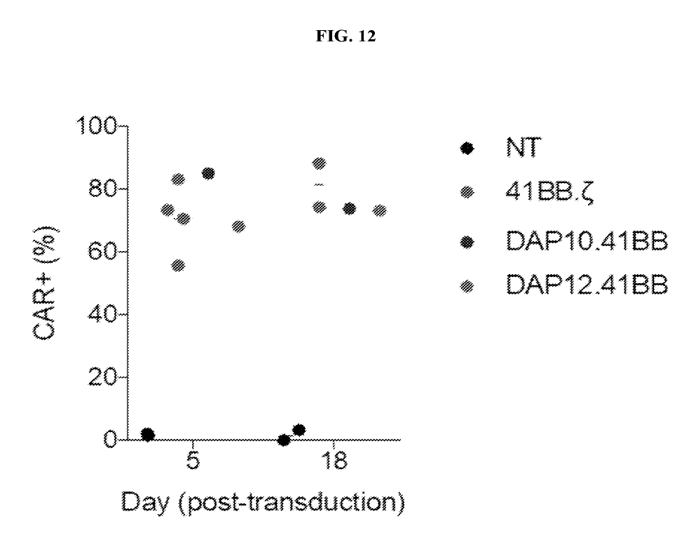

Experiments were conducted during development of embodiments herein to demonstrate the CAR modification of NK cells (FIG. 12). Healthy donor NK cells were selected from peripheral blood mononuclear cells and transduced with retroviral vectors encoding chimeric receptors. Receptors contain an ectodomain to bind CD123 and an endodomain consisting of a combination of: TCR, DAP10, DAP12, 41BB, and/or CD28. CAR expression measured on D5 and D18 post-transduction with FACS analysis.

### Example 9

### Demonstration of Specific NK-Cell Activation

Experiments were conducted during development of embodiments herein to demonstrate activation by CAR-NK cells.

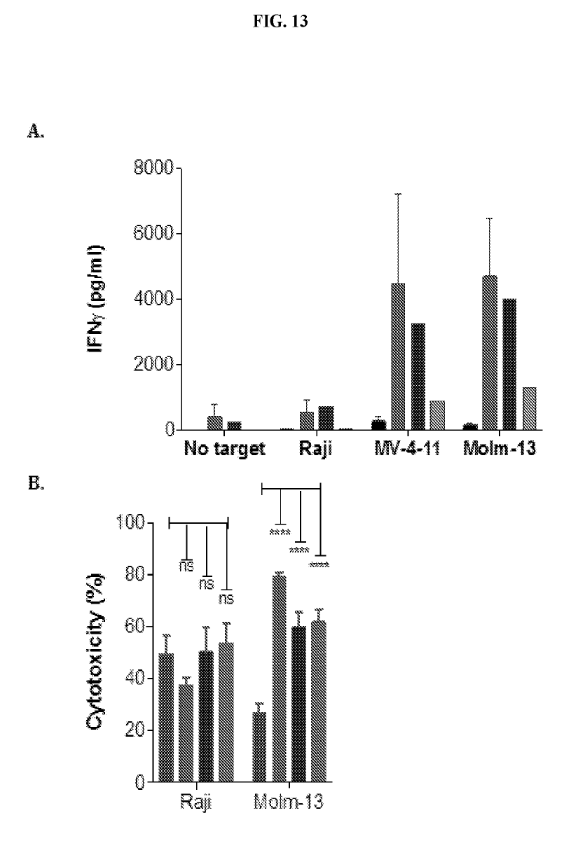

Target cells were plated in co-culture with unmodified, 41BB.ζ, DAP10.41BB, or DAP12.41BB CAR-NK cells. After 24 hours of culture, supernatant was harvested and assessed for the presence of Interferon-gamma as a marker of NK cell activation. Significant NK cell activation was measured in MV-4-11 and Molm-13 target cells (FIG. 13A).

NK cells and target (stably expressing firefly luciferase) were co-cultured for 18 hours, and bioluminescent imaging (BLI) was measured as a correlate to living cells in culture. Cytoxicity was then calculated as a comparison between cells identically treated without NK cells present (FIG. 13B). No difference was seen in cytoxicity of unmodified NK or NK-CAR for Raji (CD123-negative). Significantly increased killing (p<0.0001) was evident when comparing unmodified to CAR-NK for Molm-13 (CD123-positive).

### Example 10

### In Vivo Activation of NK-CAR

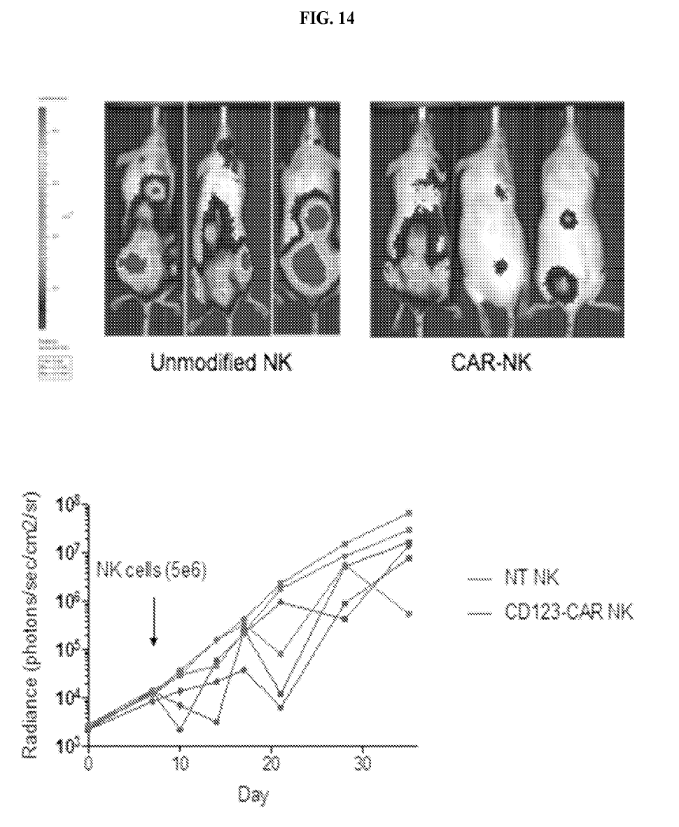

Experiments were conducted during development of embodiments herein to demonstrate in vivo activation of NK-CAR. Mice were administered leukemia cells on day 0 followed by no treatment or 5e6 CD123-CAR NK cells on day 7. Leukemia progression was monitored with detection of whole bioluminescence per mouse. Representative images of mice on day 28 post-leukemia injection are shown (FIG. 14). Line graph is representative of bioluminescence measured per mouse on indicated day post-leukemia injection (FIG. 14).

### Example 11

### Dual-NK Modification

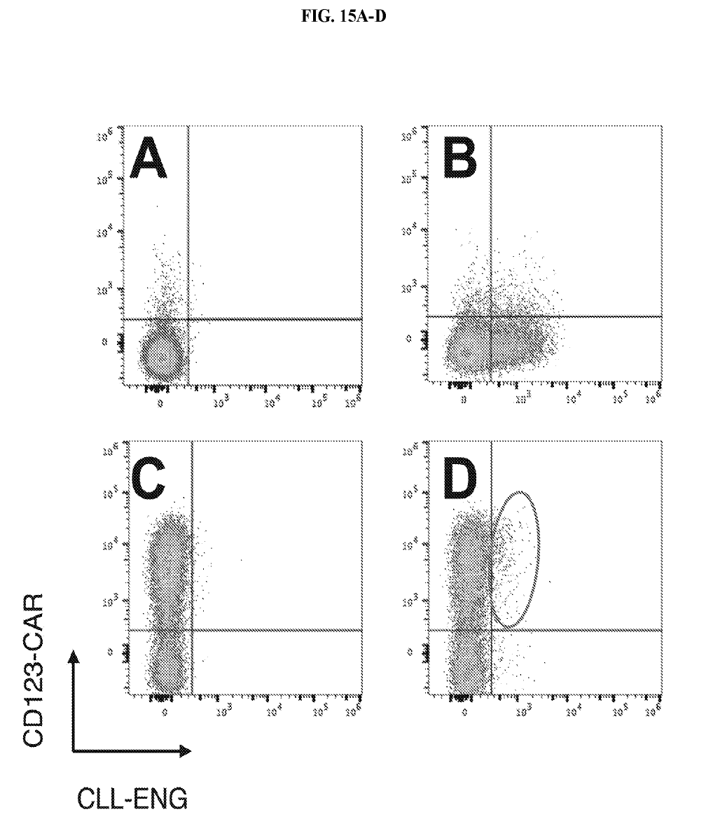

Experiments were conducted during development of embodiments herein to demonstrate dual-NK modification (FIG. 15). NK cells were transduced with retroviral vectors containing either CLL-ENG paired with the fluorophore mOrange, or a CD123 chimeric receptor. Successful genetic modification was evaluated with FACS analysis and detection of mOrange and/or CAR expression.

All publications and patents provided herein incorporated by reference in their entireties. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### REFERENCES

The following references are herein incorporated by reference in their entireties:
1. Lowenberg, B., Downing, J. R. & Burnett, A. Acute myeloid leukemia. N Engl J Med 341, 1051-1062, doi:10.1056/NEJM199909303411407 (1999).
2. Woods, W. G. Curing childhood acute myeloid leukemia (AML) at the half-way point: promises to keep and miles to go before we sleep. Pediatr Blood Cancer 46, 565-569, doi:10.1002/pbc.20646 (2006).
3. Gorman, M. F. et al. Outcome for children treated for relapsed or refractory acute myelogenous leukemia (rAML): a Therapeutic Advances in Childhood Leukemia (TACL) Consortium study. Pediatr Blood Cancer 55, 421-429, doi:10.1002/pbc.22612 (2010).
4. Kalos, M. et al. T cells with chimeric antigen receptors have potent antitumor effects and can establish memory in patients with advanced leukemia. Sci Transl Med 3, 95ra73, doi: 10.1126/scitranslmed. 3002842 (2011).
5. Kochenderfer, J. N. et al. B-cell depletion and remissions of malignancy along with cytokineassociated toxicity in a clinical trial of anti-CD19 chimeric-antigen-receptor-transduced T cells. Blood 119, 2709-2720, doi:10.1182/blood-2011-10-384388 (2012).
6. Kochenderfer, J. N. et al. Donor-derived CD19-targeted T cells cause regression of malignancy persisting after allogeneic hematopoietic stem cell transplantation. Blood 122, 4129-4139, doi:10.1182/blood-2013-08-519413 (2013).
7. Brentjens, R. J. et al. CD19-targeted T cells rapidly induce molecular remissions in adults with chemotherapy-refractory acute lymphoblastic leukemia. Sci Transl Med 5, 177ra138, doi:10.1126/scitranslmed.3005930 (2013).
8. Maude, S. L. et al. Chimeric antigen receptor T cells for sustained remissions in leukemia. N Engl J Med 371, 1507-1517, doi:10.1056/NEJMoa1407222 (2014).
9. Kochenderfer, J. N. et al. Chemotherapy-refractory diffuse large B-cell lymphoma and indolent B-cell malignancies can be effectively treated with autologous T cells expressing an anti-CD19 chimeric antigen receptor. J Clin Oncol 33, 540-549, doi:10.1200/JCO.2014.56.2025 (2015).
10. Lee, D. W. et al. T cells expressing CD19 chimeric antigen receptors for acute lymphoblastic leukaemia in children and young adults: a phase 1 dose-escalation trial. Lancet 385, 517-528, doi:10.1016/S0140-6736(14)61403-3 (2015).
11. Grupp S A et al. T cells engineered with a chimeric antigen receptor (CAR) targeting CD19 (CTL019) have long term persistence and induce durable remissions in children with relapsed, refractory ALL. Blood 124 (2014).
12. Park J H et al. Efficacy and safety of CD19-targeted 19-28z CAR modified T cells in adult patients with relapsed or refractory B-ALL. J Clin Oncol 33 (2015).
13. Sotillo, E. et al. Convergence of Acquired Mutations and Alternative Splicing of CD19 Enables Resistance to CART-19 Immunotherapy. Cancer Discov 5, 1282-1295, doi:10.1158/2159-8290.CD-15-1020 (2015).
14. Munoz, L. et al. Interleukin-3 receptor alpha chain (CD123) is widely expressed in hematologic malignancies. Haematologica 86, 1261-1269 (2001).
15. Ehninger, A. et al. Distribution and levels of cell surface expression of CD33 and CD123 in acute myeloid leukemia. Blood Cancer J 4, e218, doi:10.1038/bcj.2014.39 (2014).
16. Testa, U., Pelosi, E. & Frankel, A. CD 123 is a membrane biomarker and a therapeutic target in hematologic malignancies. Biomark Res 2, 4, doi:10.1186/2050-7771-2-4 (2014).
17. Mardiros, A. et al. T cells expressing CD123-specific chimeric antigen receptors exhibit specific cytolytic effector functions and antitumor effects against human acute myeloid leukemia. Blood 122, 3138-3148, doi:10.1182/blood-2012-12-474056 (2013).
18. Tettamanti, S. et al. Targeting of acute myeloid leukaemia by cytokine-induced killer cells redirected with a novel CD123-specific chimeric antigen receptor. Br J Haematol 161, 389-401, doi:10.1111/bjh.12282 (2013).
19. Pizzitola, I. et al. Chimeric antigen receptors against CD33/CD123 antigens efficiently target primary acute myeloid leukemia cells in vivo. Leukemia 28, 1596-1605, doi:10.1038/leu.2014.62 (2014).
20. Gill, S. et al. Preclinical targeting of human acute myeloid leukemia and myeloablation using chimeric antigen receptor-modified T cells. Blood 123, 2343-2354, doi:10.1182/blood-2013-09-529537 (2014).
21. Kenderian, S. S. et al. CD33-specific chimeric antigen receptor T cells exhibit potent preclinical activity against human acute myeloid leukemia. Leukemia 29, 1637-1647, doi:10.1038/leu.2015.52 (2015).
22. van Rhenen, A. et al. The novel AML stem cell associated antigen CLL-1 aids in discrimination between normal and leukemic stem cells. Blood 110, 2659-2666, doi:10.1182/blood-2007-03-083048 (2007).
23. Larsen, H. O., Roug, A. S., Just, T., Brown, G. D. & Hokland, P. Expression of the hMICL in acute myeloid leukemia-a highly reliable disease marker at diagnosis and during follow-up. Cytometry B Clin Cytom 82, 3-8, doi:10.1002/cyto.b.20614 (2012).
24. Roug, A. S. et al. hMICL and CD123 in combination with a CD45/CD34/CD117 backbone—a universal marker combination for the detection of minimal residual disease in acute myeloid leukaemia. Br J Haematol 164, 212-222, doi:10.1111/bjh.12614 (2014).
25. Hegde, M. et al. Combinational targeting offsets antigen escape and enhances effector functions of adoptively transferred T cells in glioblastoma. Mol Ther 21, 2087-2101, doi:10.1038/mt.2013.185 (2013).
26. Kloss, C. C., Condomines, M., Cartellieri, M., Bachmann, M. & Sadelain, M. Combinatorial antigen recognition with balanced signaling promotes selective tumor eradication by engineered T cells. Nat Biotechnol 31, 71-75, doi:10.1038/nbt.2459 (2013).
27. Wu, C. Y., Roybal, K. T., Puchner, E. M., Onuffer, J. & Lim, W. A. Remote control of therapeutic T cells through a small molecule-gated chimeric receptor. Science 350, aab4077, doi:10.1126/science.aab4077 (2015).
28. Roybal, K. T. et al. Precision Tumor Recognition by T Cells With Combinatorial Antigen-Sensing Circuits. Cell 164, 770-779, doi:10.1016/j.cell.2016.01.011 (2016).
29. Stephan, M. T. et al. T cell-encoded CD80 and 4-1BBL induce auto- and transcostimulation, resulting in potent tumor rejection. Nat Med 13, 1440-1449, doi:10.1038/nm1676 (2007).
30. Hoyos, V. et al. Engineering CD19-specific T lymphocytes with interleukin-15 and a suicide gene to enhance their anti-lymphoma/leukemia effects and safety. Leukemia 24, 1160-1170, doi:10.1038/leu.2010.75 (2010).
31. Pegram, H. J. et al. Tumor-targeted T cells modified to secrete IL-12 eradicate systemic tumors without need for prior conditioning. Blood 119, 4133-4141, doi:10.1182/blood-2011-12-400044 (2012).
32. Curran, K. J. et al. Enhancing antitumor efficacy of chimeric antigen receptor T cells through constitutive CD40L expression. Mol Ther 23, 769-778, doi:10.1038/mt.2015.4 (2015).
33. Mack, M., Riethmuller, G. & Kufer, P. A small bispecific antibody construct expressed as a functional single-chain molecule with high tumor cell cytotoxicity. Proc Natl Acad Sci USA 92, 7021-7025 (1995).
34. Topp, M. S. et al. Safety and activity of blinatumomab for adult patients with relapsed or refractory B-precursor acute lymphoblastic leukaemia: a multicentre, single-arm, phase 2 study. Lancet Oncol 16, 57-66, doi:10.1016/S1470-2045(14)71170-2 (2015).
35. Topp, M. S. et al. Long-term follow-up of hematologic relapse-free survival in a phase 2 study of blinatumomab in patients with MRD in B-lineage ALL. Blood 120, 5185-5187, doi:10.1182/blood-2012-07-441030 (2012).
36. Topp, M. S. et al. Phase II trial of the anti-CD19 bispecific T cell-engager blinatumomab shows hematologic and molecular remissions in patients with relapsed or refractory B-precursor acute lymphoblastic leukemia. J Clin Oncol 32, 4134-4140, doi:10.1200/JCO.2014.56.3247 (2014).
37. Teachey, D. T. et al. Cytokine release syndrome after blinatumomab treatment related to abnormal macrophage activation and ameliorated with cytokine-directed therapy. Blood 121, 5154-5157, doi:10.1182/blood-2013-02-485623 (2013).
38. Iwahori, K. et al. Engager T cells: a new class of antigen-specific T cells that redirect bystander T cells. Mol Ther 23, 171-178, doi:10.1038/mt.2014.156 (2015).
39. Bonifant, C. L. et al. CD123-Engager T cells as a Novel Immunotherapeutic for Acute Myeloid Leukemia. submitted.
40. Zal, T. & Chodaczek, G. Intravital imaging of anti-tumor immune response and the tumor microenvironment. Semin Immunopathol 32, 305-317, doi:10.1007/s00281-010-0217-9 (2010).
41. Savoldo, B. et al. CD28 costimulation improves expansion and persistence of chimeric antigen receptor-modified T cells in lymphoma patients. J Clin Invest 121, 1822-1826, doi:10.1172/JC146110 (2011).
42. Du, X., Ho, M. & Pastan, I. New immunotoxins targeting CD123, a stem cell antigen on acute myeloid leukemia cells. J Immunother 30, 607-613, doi:10.1097/CJI.0b013e318053ed8e (2007).
43. Abo, A. & Korver, W. Antibodies to CLL-1. U.S. Pat. No. 8,536,310 B2 (2013).
44. Mandl, J. N. et al. Quantification of lymph node transit times reveals differences in antigen surveillance strategies of naive CD4+ and CD8+ T cells. Proc Natl Acad Sci USA 109, 18036-18041, doi:10.1073/pnas.1211717109 (2012).
45. Huang, A. Y. et al. Viewing transplantation immunology through today's lens: new models, new imaging, and new insights. Biol Blood Marrow Transplant 19, S44-51, doi:10.1016/j.bbmt.2012.10.020 (2013).
46. Myers, J. T., Barkauskas, D. S. & Huang, A. Y. Dynamic Imaging of Marrow-Resident Granulocytes Interacting with Human Mesenchymal Stem Cells upon Systemic Lipopolysaccharide Challenge. Stem Cells Int 2013, 656839, doi:10.1155/2013/656839 (2013).
47. Wang, W. et al. Aberrant Notch signaling in the bone marrow microenvironment of acute lymphoid leukemia suppresses osteoblast-mediated support of hematopoietic niche function. Cancer Res, doi:10.1158/0008-5472.CAN-15-2092 (2016).

1. A method of treating acute myeloid leukemia (AML) in a subject, the method comprising adoptively transferring engineered lymphocytes that express a polypeptide construct comprising an antigen-recognition domain and an activation domain, wherein the antigen-recognition domain binds C-type lectin-like molecule-1 (CLL-1) displayed on a malignant myeloblast cell, and wherein the activation domain triggers an immune response against the malignant myeloblast cell by the engineered lymphocytes and/or native T cells upon binding of the antigen-recognition domain to CLL-1.

2. The method of claim 1, wherein the polypeptide construct is a bispecific engager molecule which is secreted from the engineered lymphocytes.

3. The method of claim 2, wherein the antigen-recognition domain is an antibody fragment that binds CLL-1.

4. The method of claim 3, wherein the antibody fragment that binds CLL-1 is a single chain variable fragment (scFv).

5. The method of claim 2, wherein the activation domain is a molecular moiety that interacts with T cell receptor (TCR) and induces an immunomodulatory signal.

6. The method of claim 5, wherein the activation domain is an antibody fragment that binds CD3.

7. The method of claim 6, wherein the antibody fragment that binds CD3 is a single chain variable fragment (scFv).

8. The method of claim 1, wherein the polypeptide construct is a single polypeptide bispecific engager molecule comprising an scFv activation domain that binds CD3 and an scFv antigen-recognition domain that binds CLL-1.

9. The method of claim 1, wherein the polypeptide construct is a chimeric antigen receptor (CAR) which is displayed on the surface of the engineered lymphocytes, and wherein the antigen-recognition domain and the activation domain are linked by a transmembrane domain.

10. The method of claim 9, wherein the antigen-recognition domain is an antibody fragment that binds CLL-1.

11. The method of claim 10, wherein the antibody fragment that binds CLL-1 is a single chain variable fragment (scFv).

12. The method of claim 9, wherein the activation domain is an intracellular signaling domain that initiates signal transduction to activate the engineered lymphocyte upon antigen binding by the antigen-recognition domain

13. The method of claim 12, wherein the activation domain comprises a cytoplasmic sequence of the T cell receptor (TCR) and or co-receptors.

14. The method of claim 1, wherein the engineered lymphocytes are engineered T-cells, engineered NK cells, or engineered NKT cells.

15. The method of claim 1, further comprising obtaining lymphocytes from the subject, genetically engineering the lymphocytes to express the polypeptide construct, and culturing the resulting engineered lymphocytes.

16. A bispecific engager molecule comprising:
(a) an antigen-recognition domain that specifically binds to C-type lectin-like molecule-1 (CLL-1); and
(b) an activation domain that interacts with a portion of T cell receptor (TCR) to induce an immunomodulatory signal.

17. The bispecific engager molecule of claim 16, wherein the antigen-recognition domain is an antibody fragment.

18. The bispecific engager molecule of claim 17, wherein the antigen-recognition domain is a single chain variable fragment (scFv).

19. The bispecific engager molecule of claim 16, wherein the activation domain is an antibody fragment.

20. The bispecific engager molecule of claim 19, wherein the activation domain is a single chain variable fragment (scFv).

21. The bispecific engager molecule of claim 16, wherein the activation domain is an anti-CD3 antibody fragment.

22. The bispecific engager molecule of claim 16, wherein the activation domain and antigen-recognition domain are single chain variable fragments tethered to each other by a linker domain.

23. An engineered lymphocyte comprising a polynucleotide that encodes a bispecific engager molecule of one of claims 16-22.

24. The engineered lymphocyte of claim 23, wherein the engineered lymphocyte expresses the bispecific engager molecule from the polynucleotide and the bispecific engager molecule is secreted from the engineered lymphocyte.

24. The engineered lymphocyte of claim 23, wherein the lymphocyte is a T cell.

25. The engineered lymphocyte of claim 23, wherein the engineered lymphocyte displays a molecular moiety on its surface that is a component of T cell receptor (TCR) or interacts with TCR to induce an immunomodulatory signal, and wherein the activation domain of the bispecific engager molecule is capable of binding the molecular moiety.

26. The engineered lymphocyte of claim 23, wherein the lymphocyte is an NK cell.

27. The engineered lymphocyte of claim 26, wherein the NK cell expresses a chimeric antigen receptor (CAR) that comprises an antigen-recognition domain that is capable of binding to a cancer cell antigen.

28. The engineered lymphocyte of claim 27, wherein the antigen-recognition domain of the CAR is capable of binding to CD123.

29. The engineered lymphocyte of claim 27, wherein the CAR comprises in intracellular signaling domain that activates an immunomodulatory signal upon binding of the antigen-recognition domain of the CAR is capable of binding to CD123.

30. An adoptive transfer method comprising administering an engineered lymphocyte of one of claims 23-29 to a subject.

31. The adoptive transfer method of claim 30, wherein the subject suffers from cancer.

32. The adoptive transfer method of claim 31, wherein the subject suffers from leukemia.

33. The adoptive transfer method of claim 32, wherein the subject suffers from acute myeloid leukemia (AML).

34. The adoptive transfer method of claim 30, wherein the engineered lymphocyte expresses and secretes the bispecific engager molecule within the subject.

35. The adoptive transfer method of claim 30, wherein the bispecific engager molecule binds to malignant cells displaying CLL-1 and T cells, thereby activating the T cells to attack the malignant cells displaying CLL-1.

36. An engineered lymphocyte comprising:
(a) a first polynucleotide sequence encoding bispecific engager molecule that comprises an antigen-recognition domain capable of binding a first antigen and an activation domain capable of binding a molecule moiety displayed on T cells that activates an immunomodulatory signal upon binding; and
(b) a second polynucleotide sequence encoding a chimeric antigen receptor (CAR) that comprises an antigen-recognition domain capable of binding a second antigen and an intracellular signaling domain that activates an immunomodulatory signal upon binding of the antigen-recognition domain to the second antigen, wherein the intracellular signaling domain and the antigen-recognition domain of the CAR are linked by a transmembrane domain.

37. The engineered lymphocyte of claim 36, wherein the first polynucleotide sequence and the second polynucleotide sequence are portions of a single nucleic acid or vector.

38. The engineered lymphocyte of claim 36, wherein the first polynucleotide sequence and the second polynucleotide sequence are portions of separate nucleic acids or vectors.

39. The engineered lymphocyte of claim 36, wherein the lymphocyte is a T cell.

40. The engineered lymphocyte of claim 39, wherein the activation domain of the bispecific engager molecule is capable of initiating primary immunomodulatory activation through T cell receptor, and the intracellular signaling domain of the CAR initiates a co-stimulatory immunomodulatory signal.

41. The engineered lymphocyte of claim 36, wherein the lymphocyte is an NK cell.

42. The engineered lymphocyte of claim 41, wherein the activation domain of the bispecific engager molecule is capable of initiating primary immunomodulatory activation through T cell receptor, and the intracellular signaling domain of the CAR initiates primary immunomodulatory activation of the NK cell.

43. The engineered lymphocyte of claim 36, wherein the first antigen-recognition domain is capable of binding CLL-1 and the second antigen-recognition domain is capable of binding CD123.

44. The engineered lymphocyte of claim 36, wherein the first antigen-recognition domain is capable of binding CD123 and the second antigen-recognition domain is capable of binding CLL-1.

45. A method of treating a disease or condition comprising administering the engineered lymphocyte of one or claims 36-44 to a subject.

46. The method of claim 45, wherein the subject suffers from cancer.

47. The method of claim 46, wherein the subject suffers from leukemia.

48. The method of claim 47, wherein the subject suffers from acute myeloid leukemia (AML).

49. Use of an engineered lymphocyte of one or claim 23-29 or 36-44 for the treatment of a disease or condition.

50. The use of claim 49, wherein the disease or condition is cancer.

51. The use of claim 50, wherein the disease or condition is leukemia.

52. The use of claim 51, wherein the disease or condition is acute myeloid leukemia (AML).
